(19) United States
(12) Patent Application Publication
(10) Pub. No.: US 2026/0138982 A1
(43) Pub. Date: May 21, 2026

(54) Biaryl Acid Compounds

(71) Applicant: Pfizer Inc., New York, NY (US)

(72) Inventors: James Lester COLLINS, III, San Diego, CA (US); Ketan Satish GAJIWALA, San Diego, CA (US); Gary Michael GALLEGO, San Diego, CA (US); Rebecca Anne GALLEGO, San Diego, CA (US); Jacqui Elizabeth HOFFMAN, San Diego, CA (US); Mehran JALAIE, San Diego, CA (US); Hanna Maria WISNIEWSKA, San Diego, CA (US)

(21) Appl. No.: 19/389,226

(22) Filed: Nov. 14, 2025

Related U.S. Application Data
(60) Provisional application No. 63/721,253, filed on Nov. 15, 2024.
(60) Provisional application No. 63/910,617, filed on Nov. 3, 2025.

(51) Int. Cl. C07D 487/04; A61K 31/437; A61K 31/444; A61K 31/506; A61K 31/519; A61K 45/06; C07D 401/14; C07D 471/04

(52) U.S. Cl. CPC C07D 487/04; A61K 31/437; A61K 31/444; A61K 31/506; A61K 31/519; A61K 45/06; C07D 401/14; C07D 471/04

(57) ABSTRACT

The present disclosure relates to biaryl acid compounds and pharmaceutically acceptable salts thereof to their use in medicine; to pharmaceutical compositions containing them; to processes for their preparation; and to intermediates used in such processes. The biaryl acid compounds may be useful in the treatment of diseases and disorders.

### BACKGROUND OF THE INVENTION

The present invention relates to novel biaryl acid compounds. The invention also relates to the preparation of the compounds and intermediates used in the preparation, compositions containing the compounds, and uses of the compounds including treatment of disorders associated with phosphatidylinositol-4,5-bisphosphate 3-kinase catalytic subunit alpha (PI3K-α) signaling of cellular proliferative disease, such as cancer.

The activity of cells can be regulated by external signals that stimulate or inhibit intracellular events. The process by which stimulatory or inhibitory signals are transmitted into and within a cell to elicit an intracellular response is referred to as signal transduction. Over the past decades, cascades of signal transduction events have been elucidated and found to play a central role in a variety of biological responses. Defects in various components of signal transduction pathways have been found to account for a vast number of diseases, including numerous forms of cancer (e.g., breast cancer, colon cancer, bladder cancer), inflammatory disorders, metabolic disorders, vascular and neuronal diseases (Gaestel, M., et al. “Protein Kinases as Small Molecule Inhibitor Targets in Inflammation.” Current Medicinal Chemistry. 14:2214-2234 (2007)).

Phosphatidylinositol 3-kinases (PI3Ks, also known as phosphoinositide 3-kinases) comprise a family of lipid kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3), which, in turn, act as second messengers in signaling cascades by docking proteins containing pleckstrin-homology, FYVE, Phox and other phospholipid-binding domains into a variety of signaling complexes often at the plasma membrane (Vanhaesebroeck, B., et al. “Synthesis and Function of 3-Phosphorylated Inositol Lipids.” Annu. Rev. Biochem. 70:535-602 (2001); Katso, R. et al. “Cellular Function of Phosphoinositide 3-Kinases: Implications for Development, Immunity, Homeostasis, and Cancer.” Annu. Rev. Cell Dev. Biol. 17:615-75 (2001)). Of the two Class 1 PI3K sub-classes, Class 1A PI3Ks are heterodimers composed of a catalytic p110 subunit (alpha, beta, or delta isoforms) constitutively associated with a regulatory subunit that can be p85 alpha, p55 alpha, p50 alpha, p85 beta, or p55 gamma. The Class 1B sub-class has one family member, a heterodimer composed of a catalytic p110 gamma subunit associated with one of two regulatory subunits, p101 or p84 (Fruman, D., et al. “Phosphoinositide kinases.” Annu. Rev. Biochem. 67:481-507 (1998); Suire, S., et al. “p84, a New Gβγ-Activated Regulatory Subunit of the Type IB Phophoinositide 3-Kinase p110γ.” Curr. Biol. 15:566-570 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class 1B PI3K is activated directly by G protein-coupled receptors that bind a diverse repertoire of peptide and non-peptide ligands (Stephens, L., et al. “The Gβγ Sensitivity of a PI3K Is Dependent upon a Tightly Associated Adaptor, p101.” Cell. 89:105-114 (1997); Katso, R., et al., supra).

Consequently, the resultant phospholipid products of Class I PI3Ks link upstream receptors with downstream cellular activities including proliferation, survival, chemotaxis, cellular trafficking, motility, metabolism, inflammatory and allergic responses, transcription and translation (Cantley, L., et al. “Oncogenes and Signal Transduction.” Cell. 64:281-302 (1991); Escobedo, J. et al. “A PDGF receptor domain essential for mitogenesis but not for many other responses to PDGF.” Nature. 335:85-87 (1988); Fantl, W., et al. “Distinct Phosphotyrosines on a Growth Factor Receptor Bind to Specific Molecules That Mediate Different Signaling Pathways.” Cell. 69:413-423 (1992)). In many cases, PIP2 and PIP3 recruit Aid, the product of the human homologue of the viral oncogene v-Akt, to the plasma membrane where it acts as a nodal point for many intracellular signaling pathways important for growth and survival (Fantl, W., et al, Id.; Bader, A., et al. “Oncogenic PI3K Deregulates Transcription and Translation.” Nature Rev. Cancer. 5:921-929 (2005); Vivanco, I., et al. “The Phosphatidylinositol 3-Kinase-AKT Pathway in Human Cancer.” Nature Rev. Cancer. 2:489-501 (2002)).

The PI3Ks signaling pathway is one of the most highly mutated systems in human cancers. PI3K signaling is also a key factor in many other diseases in humans. PI3K signaling is involved in many disease states including allergic contact dermatitis, rheumatoid arthritis, osteoarthritis, inflammatory bowel diseases, chronic obstructive pulmonary disorder, psoriasis, multiple sclerosis, asthma, disorders related to diabetic complications, and inflammatory complications of the cardiovascular system such as acute coronary syndrome.

Aberrant regulation of PI3K has been shown to occur at multiple levels. The tumor suppressor gene PTEN, which dephosphorylates phosphoinositides at the 3′ position of the inositol ring, and in so doing antagonizes PI3K activity, is functionally deleted in a variety of tumors. In other tumors, the genes for the pi 10 alpha isoform, PIK3CA, and for Akt are amplified, and increased protein expression of their gene products has been demonstrated in several human cancers. Furthermore, mutations and translocation of p85 alpha that serve to up-regulate the p85-pl 10 complex have been described in human cancers. Finally, somatic missense mutations in PIK3CA that activate downstream signaling pathways have been described at significant frequencies in a wide diversity of human cancers (Kang, S. et al. “Phosphatidylinositol 3-kinase mutations in human cancer are oncogenic.” Proc. Natl. Acad. Sci. 102:802-807 (2005); Samuels, Y., et al. “High Frequency of Mutations of the PIK3CA Gene in Human Cancers.” Science. 304:554 (2004); Samuels, Y. et al. “Mutant PIK3CA promotes cell growth and invasion of human cancer cells.” Cancer Cell. 7:561-573 (2005)). These observations show that deregulation of phosphoinositol-3 kinase, and the upstream and downstream components of this signaling pathway, is one of the most common deregulations associated with human cancers and proliferative diseases (Parsons, D., et al. “Mutations in a signalling pathway.” Nature. 436:792 (2005); Hennessy, B., et al. “Exploiting the PI3K/AKT Pathway For Cancer Drug Discovery.” Nature Rev. Drug Disc. 4:988-1004 (2005)).

In view of the above, inhibitors of PI3K-α would be of particular value in the treatment of proliferative disease and other disorders. While multiple inhibitors of PI3Ks have been developed (for example, taselisib, alpelisib, buparlisib and others), these molecules inhibit multiple Class 1A PI3K isoforms. Inhibitors that are active against multiple Class 1A PI3K isoforms are known as “pan-PI3K” inhibitors. A major hurdle for the development of PI3K pathway inhibitors has been the inability to achieve optimal drug target blockade in tumors while avoiding undue toxicities in patients. Pan-PI3K inhibitors share common, dose-dependent toxicities such as rash, fatigue, hyperglycemia, and diarrhea. In general, toxicity from small-molecule PI3K wild-type inhibitors depends on their PI3K isozyme specificity. For example, PI3Kα inhibitors are associated with hyperglycemia and rash, whereas PI3Ko inhibitors are associated with gastrointestinal side effects, myelosuppression, and transaminitis. (Hanker, A., et al. “Challenges for the Clinical Development of PI3K Inhibitors: Strategies to Improve Their Impact in Solid Tumors.” Cancer Discovery. 9 (4): 482-491 (2019)).

As such, tolerability and safety are important considerations in structuring courses of treatment for many diseases. For example, treatments using therapeutic agents that result in severe adverse events may become ineffective clinically due to insufficient patient compliance or because an effective therapeutic dose cannot be safely administered to the patient.

Accordingly, there remains a need for maximizing therapeutic benefit, while ameliorating drug-related toxicities. To this end development of mutant PI3K-α selective inhibitors, methods of the present invention, along with selecting patients with PI3K-α mutations should improve the success of these targeted compounds. More importantly, these selective compounds will be particularly important in cancer treatment where treatment times can be long. Accordingly, mutant PI3K-α selective inhibitors of PI3K-α may increase the therapeutic window, enabling sufficient target inhibition in the tumor while avoiding dose-limiting toxicity in cancer patients.

### SUMMARY OF THE INVENTION

The present invention provides, in part, compounds of Formula (I) and pharmaceutically acceptable salts thereof. The compounds of the present disclosure may inhibit the activity of PI3K-α and mutations thereof and may be useful in the treatment, prevention, suppression and amelioration of diseases, disorders or conditions associated with PI3K-α signaling and mutations thereof of cellular proliferative disease, such as cancer. Also provided are pharmaceutical compositions, comprising the compounds or salts of the invention, alone or in combination with additional therapies, such as radiation therapy and/or chemotherapy. The present invention also provides, in part, methods for preparing such compounds, pharmaceutically acceptable salts and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the disclosure, there is provided a compound of Formula (I):

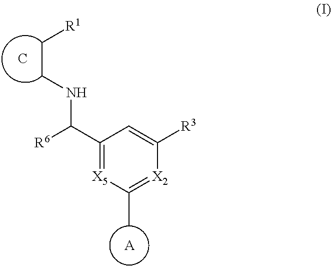

or a pharmaceutically acceptable salt thereof, wherein:
Ring A is a nine or ten membered bicyclic heteroaryl containing one, two or three heteroatoms selected from the group consisting of N, O and S, wherein the heteroaryl is optionally substituted with one or two groups independently selected from the group consisting of halogen, C₁-C₆ alkyl, C₁-C₆ alkoxy, C₃-C₆ cycloalkyl, and NR^{a}R^{b};
Ring C is phenyl or a 5 or 6 membered heteroaryl containing one or two heteroatoms independently selected from N and S, wherein Ring C is optionally substituted by R²;
X₂ and X₅ are independently selected from the group consisting of N and CH;
R¹ is selected from the group consisting of —C(═O)OH and a carboxylic acid isostere;
R² is selected from the group consisting of hydrogen, halogen, C₁-C₃ alkyl, and C₁-C₃ fluoroalkyl;
R³ is selected from the group consisting of halogen, C₁-C₃ alkyl, and C₁-C₃ haloalkyl;
R⁶ is hydrogen or methyl; and
R^{a} and R^{b} are independently selected from hydrogen and C₁-C₃ alkyl.

Described below are embodiments of the invention, where for convenience Embodiment 1 (E1) is identical to the embodiment of Formula (I) provided above.

It is to be understood that both the foregoing general description and the following detailed description are exemplary and explanatory only and are not restrictive of the invention, as claimed.

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.
E1 A compound of Formula (I) or a pharmaceutically acceptable salt thereof, as defined above.
E2 A compound of embodiment E1, or a pharmaceutically acceptable salt thereof, wherein X₂ is N.
E3 A compound of embodiment E1, or a pharmaceutically acceptable salt thereof, wherein X₂ is CH.
E4 A compound of any one of embodiments E1 to E3, or a pharmaceutically acceptable salt thereof, wherein X₅ is N.
E5 A compound of any one of embodiments E1 to E3, or a pharmaceutically acceptable salt thereof, wherein X₅ is CH.
E6 A compound of any one of embodiments E1 or E2, or a pharmaceutically acceptable salt thereof, wherein X₂ is N and X₅ is CH.
E7 A compound of any one of embodiments E1 or E2, or a pharmaceutically acceptable salt thereof, wherein X₂ is N and X₅ is N.
E8 A compound of any one of embodiments E1 or E3, or a pharmaceutically acceptable salt thereof, wherein X₂ is CH and X₅ is CH.
E9 A compound of any one of embodiments E1 or E3, or a pharmaceutically acceptable salt thereof, wherein X₂ is CH and X₅ is N.
E10 A compound of any one of embodiments E1 to E9, or a pharmaceutically acceptable salt thereof, wherein R³ is selected from the group consisting of halogen, methyl, and trifluoromethyl.
E11 A compound of any one of embodiments E1 to E10, or a pharmaceutically acceptable salt thereof, wherein R³ is methyl.
E12 A compound of any one of embodiments E1 to E11, or a pharmaceutically acceptable salt thereof, wherein R⁶ is methyl.
E13 A compound of embodiment E1, or a pharmaceutically acceptable salt thereof, wherein the compound has the structure of Formula (Ia):

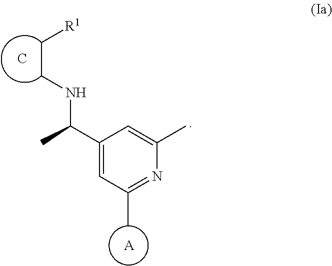

E14 A compound of any one of embodiments E1 to E13, or a pharmaceutically acceptable salt thereof, wherein Ring A is a nine or ten membered bicyclic heteroaryl containing two or three N heteroatoms, wherein the heteroaryl is optionally substituted with one or two groups independently selected from the group consisting of halogen, C₁-C₆ alkyl, C₁-C₆ alkoxy, C₃-C₆ cycloalkyl, NH₂ and NH(CH₃).
E15 A compound of any one of embodiments E1 to E14, or a pharmaceutically acceptable salt thereof, wherein Ring A is a nine-membered bicyclic heteroaryl containing two or three N heteroatoms, wherein the heteroaryl is optionally substituted with one or two groups independently selected from the group consisting of halogen, C₁-C₆ alkyl, C₁-C₆ alkoxy, and C₃-C₆ cycloalkyl.
E16 A compound of any one of embodiments E1 to E13, or a pharmaceutically acceptable salt thereof, wherein Ring A is selected from the group consisting of:

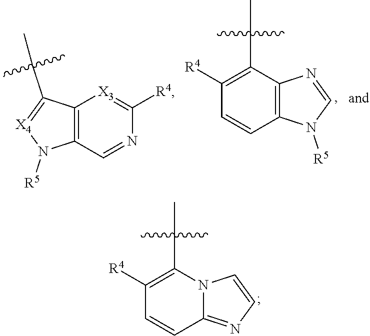

wherein X₃ and X₄ are independently selected from N and CH;
R⁴ is selected from the group consisting of selected from the group consisting of hydrogen, halogen, C₁-C₆ alkyl, C₁-C₆ alkoxy, NH₂ and NH(CH₃); and
R⁵ is selected from the group consisting of hydrogen, C₁-C₆ alkyl, and C₃-C₆ cycloalkyl.
E17 A compound of embodiment E16, or a pharmaceutically acceptable salt thereof, wherein X₃ and X₄ are independently selected from N and CH, wherein only one of X₃ and X₄ may be N.
E18 A compound of embodiment E17, or a pharmaceutically acceptable salt thereof, wherein X₃ is N and X₄ is CH.
E19 A compound of embodiment E17, or a pharmaceutically acceptable salt thereof, wherein X₃ is CH and X₄ is N.
E20 A compound of embodiment E17, or a pharmaceutically acceptable salt thereof, wherein X₃ is CH and X₄ is CH.
E21 A compound of embodiment E17, or a pharmaceutically acceptable salt thereof, wherein Ring A is:

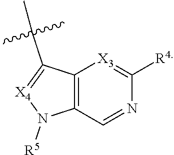

E22 A compound of any one of embodiments E16 to E121, or a pharmaceutically acceptable salt thereof, wherein R⁴ is selected from the group consisting of hydrogen, halogen, C₁-C₆ alkyl, and C₁-C₆ alkoxy. The further embodiment, wherein R⁴ is selected from the group consisting of hydrogen, chloro, methyl, and methoxy.
E23 A compound of any one of embodiments E16 to E22, or a pharmaceutically acceptable salt thereof, wherein R⁵ is selected from the group consisting of hydrogen, methyl, ethyl, isopropyl, and cyclopentyl.
E24 A compound of any one of embodiments E1 to E12 and E14 to E23, or a pharmaceutically acceptable salt thereof, wherein the compound has the structure of Formula (II):

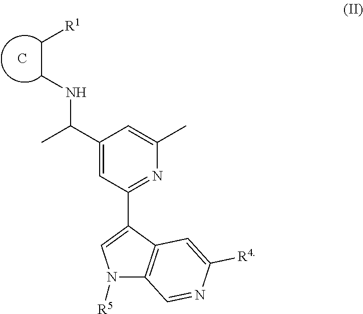

E25 A compound of any one of embodiments E1 to E24, or a pharmaceutically acceptable salt thereof, wherein the compound has the structure of Formula (IIa):

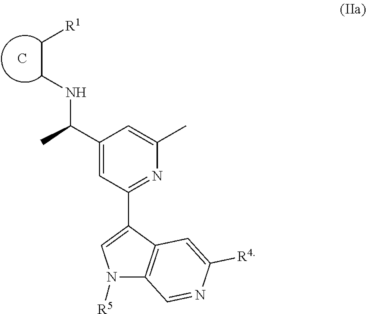

Ring C is a phenyl or a 5 or 6 membered heteroaryl containing one or two heteroatoms independently selected from N and S, wherein Ring C is optionally substituted by R². Ring C bonded to the NH moiety of Formula I via a carbon and contains an adjacent carbon substituted by R¹ as shown by the structure:

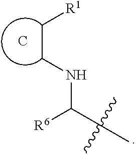

Ring C contains an optional substitution of R² which may be bonded to a ring carbon or ring nitrogen atom.
E26 A compound of any one of embodiments E1 to E25, or a pharmaceutically acceptable salt thereof, wherein Ring C is selected from the group consisting of phenyl, pyridine, pyrazole, and thiazole, wherein Ring C is optionally substituted by R².
E27 A compound of any one of embodiments E1 to E26, or a pharmaceutically acceptable salt thereof, wherein Ring C is selected from the group consisting of:

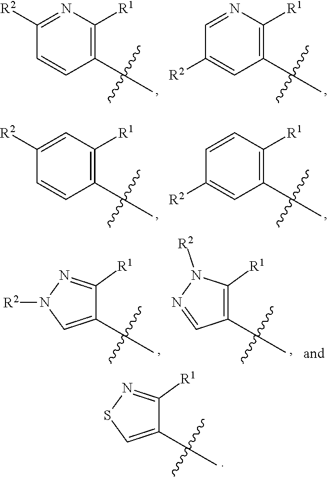

The further embodiment, wherein Ring C is selected from the group consisting of:

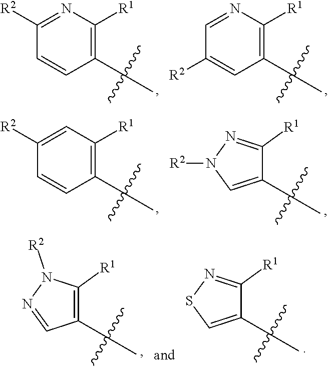

E28 A compound of any one of embodiments E1 to E27, or a pharmaceutically acceptable salt thereof, wherein R² is selected from the group consisting of hydrogen, Cl, F, methyl, and difluoromethyl.
E29 A compound of embodiment E1, or a pharmaceutically acceptable salt thereof, having the structure of Formula (I′):

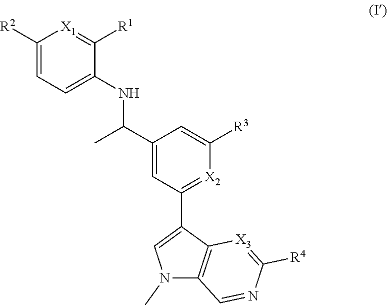

wherein X₁ and X₃ are independently selected from the group consisting of N and CH; and R⁴ is selected from the group consisting of hydrogen, halogen, methyl, methoxy, NH₂, and NH(CH₃).
E30 A embodiment of E29 or a pharmaceutically acceptable salt thereof, having the Formula (Ia′):

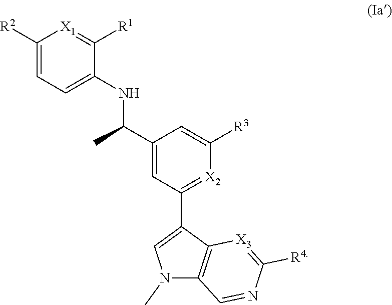

E31 A compound of any one of embodiments E29 or E30, or a pharmaceutically acceptable salt thereof, wherein R² is hydrogen or Cl.
E32 A compound of any one of embodiments E29 to E31, or a pharmaceutically acceptable salt thereof, wherein R³ is methyl.
E33 A compound of any one of embodiments E29 to E32, or a pharmaceutically acceptable salt thereof, wherein R⁴ is hydrogen or Cl.
E34 A compound of any one of embodiments E29 to E33, or a pharmaceutically acceptable salt thereof, wherein X₃ is CH.
E35 A compound of embodiment E1, having Formula (II′):

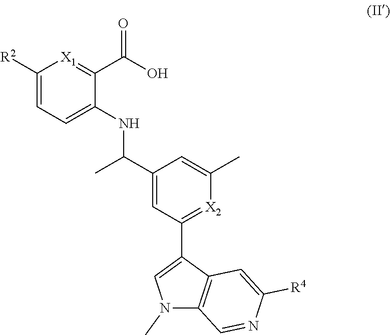

wherein X₁ is selected from the group consisting of N and CH; and
R⁴ is selected from the group consisting of hydrogen, halogen, methyl, methoxy, NH₂, and NH(CH₃);
or a pharmaceutically acceptable salt thereof.
E36 A compound of embodiment E35, or a pharmaceutically acceptable salt thereof, having Formula (Ila′):

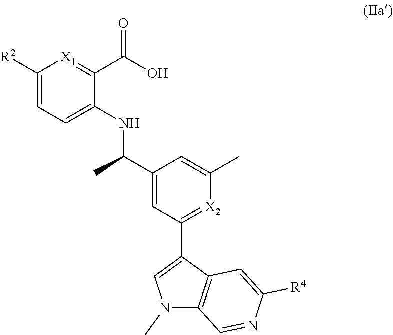

or a pharmaceutically acceptable salt thereof.
E37 A compound of any one of embodiments E1, E35, or E36, or a pharmaceutically acceptable salt thereof, wherein R² is hydrogen or Cl.
E38 A compound of any one of embodiments E1 or E35 to E37, or a pharmaceutically acceptable salt thereof, wherein R⁴ is hydrogen or Cl.
   R¹ is selected from the group consisting of —C(═O) OH and a carboxylic acid isostere. Carboxylic acid isosteres are known to those of skill in the art, for example, see Lassalas, Pierrik, “Structure Property Relationships of Carboxylic Acid Isosteres.” J. Med. Chem. 2016, 59, 7, 3183-3203.
E39 A compound of any one of embodiments E1 to E34, or a pharmaceutically acceptable salt thereof, wherein R¹ is selected from the group consisting of —C(═O)OH, —C(═O)NH₂, —C(═O)NHSO₂CH₃, —NHC(═O)NHSO₂CH₃, tetrazole, —SO₂NH₂, —NHSO₂CH₃, and 5-oxo-1,2,4-oxadizole. The further embodiment wherein R¹ is selected from the group consisting of —C(═O)OH, —C(═O)NH₂, and —C(═O)NHSO₂CH₃. A compound of any one of embodiments E1 to E34, or a pharmaceutically acceptable salt thereof, wherein R¹ is —C(═O)OH.
E40 A compound of embodiment E1 selected from the group consisting of:
3-({(1R*)-1-[2-(5-chloro-1-methyl-1H-pyrrolo[2,3-c]pyridin-3-yl)-6-methylpyridin-4-yl]ethyl}amino)pyridine-2-carboxylic acid;
2-({(1R*)-1-[2-methyl-6-(1-methyl-1H-pyrrolo[2,3-c]pyridin-3-yl) pyridin-4-yl]ethyl}amino)benzoic acid;
2-({(1R*)-1-[3-methyl-5-(1-methyl-1H-pyrrolo[2,3-c]pyridin-3-yl)phenyl]ethyl}amino)benzoic acid;
3-({(1R*)-1-[3-methyl-5-(1-methyl-1H-pyrrolo[2,3-c]pyridin-3-yl)phenyl]ethyl}amino)pyridine-2-carboxylic acid;
6-chloro-3-({(1R*)-1-[3-methyl-5-(1-methyl-1H-pyrrolo[2,3-c]pyridin-3-yl)phenyl]ethyl}amino)pyridine-2-carboxylic acid; and
3-({(1R*)-1-[2-methyl-6-(1-methyl-1H-pyrrolo[2,3-c]pyridin-3-yl) pyridin-4-yl]ethyl}amino)pyridine-2-carboxylic acid;
   or a pharmaceutically acceptable salt thereof.
   E41 A compound of embodiment E1 selected from the group consisting of:
3-({(1R*)-1-[2-(5-chloro-1-methyl-1H-pyrrolo[2,3-c]pyridin-3-yl)-6-methylpyridin-4-yl]ethyl}amino)pyridine-2-carboxylic acid;
3-({(1S*)-1-[2-(5-chloro-1-methyl-1H-pyrrolo[2,3-c]pyridin-3-yl)-6-methylpyridin-4-yl]ethyl}amino)pyridine-2-carboxylic acid;
2-({(1R*)-1-[2-methyl-6-(1-methyl-1H-pyrrolo[2,3-c]pyridin-3-yl) pyridin-4-yl]ethyl}amino)benzoic acid;
2-({(1S*)-1-[2-methyl-6-(1-methyl-1H-pyrrolo[2,3-c]pyridin-3-yl) pyridin-4-yl]ethyl}amino)benzoic acid;
2-({(1R*)-1-[3-methyl-5-(1-methyl-1H-pyrrolo[2,3-c]pyridin-3-yl)phenyl]ethyl}amino)benzoic acid;
2-({(1S*)-1-[3-methyl-5-(1-methyl-1H-pyrrolo[2,3-c]pyridin-3-yl)phenyl]ethyl}amino)benzoic acid;
3-({(1R*)-1-[3-methyl-5-(1-methyl-1H-pyrrolo[2,3-c]pyridin-3-yl)phenyl]ethyl}amino)pyridine-2-carboxylic acid;
3-({(1S″)-1-[3-methyl-5-(1-methyl-1H-pyrrolo[2,3-c]pyridin-3-yl)phenyl]ethyl}amino)pyridine-2-carboxylic acid;
6-chloro-3-({(1R*)-1-[3-methyl-5-(1-methyl-1H-pyrrolo[2,3-c]pyridin-3-yl)phenyl]ethyl}amino)pyridine-2-carboxylic acid;
6-chloro-3-({(1S*)-1-[3-methyl-5-(1-methyl-1H-pyrrolo[2,3-c]pyridin-3-yl)phenyl]ethyl}amino)pyridine-2-carboxylic acid;
3-({(1R*)-1-[2-methyl-6-(1-methyl-1H-pyrrolo[2,3-c]pyridin-3-yl) pyridin-4-yl]ethyl}amino)pyridine-2-carboxylic acid;
3-({(1S*)-1-[2-methyl-6-(1-methyl-1H-pyrrolo[2,3-c]pyridin-3-yl) pyridin-4-yl]ethyl}amino)pyridine-2-carboxylic acid;
4-({1-[2-(5-chloro-1-methyl-1H-pyrrolo[2,3-c]pyridin-3-yl)-6-methylpyridin-4-yl]ethyl}amino)-1-methyl-1H-pyrazole-3-carboxylic acid;
3-({(1R)-1-[2-(5-chloro-1-methyl-1H-pyrrolo[2,3-c]pyridin-3-yl)-6-methylpyridin-4-yl]ethyl}amino)-6-methylpyridine-2-carboxylic acid;
2-({(1R*)-1-[3-(5-methoxy-1-methyl-1H-pyrrolo[2,3-c]pyridin-3-yl)-5-methylphenyl]ethyl}amino)benzoic acid;
2-({(1S*)-1-[3-(5-methoxy-1-methyl-1H-pyrrolo[2,3-c]pyridin-3-yl)-5-methylphenyl]ethyl}amino)benzoic acid;
N-(methanesulfonyl)-2-({(1R)-1-[3-methyl-5-(1-methyl-1H-pyrrolo[2,3-c]pyridin-3-yl)phenyl]ethyl}amino)benzamide;
2-({(1R)-1-[2-(5-chloro-1-methyl-1H-pyrrolo[2,3-c]pyridin-3-yl)-6-methylpyridin-4-yl]ethyl}amino)-5-fluorobenzoic acid;
3-({[2-(5-chloro-1-methyl-1H-pyrrolo[2,3-c]pyridin-3-yl)-6-methylpyridin-4-yl]methyl}amino)pyridine-2-carboxylic acid;
3-({(1R)-1-[2-(5-chloro-1-methyl-1H-pyrrolo[2,3-c]pyridin-3-yl)-6-methylpyridin-4-yl]ethyl}amino)pyridine-2-carboxamide;
4-({1-[2-(5-chloro-1-methyl-1H-pyrrolo[2,3-c]pyridin-3-yl)-6-methylpyridin-4-yl]ethyl}amino)-1-methyl-1H-pyrazole-5-carboxylic acid;
3-({1-[2-(5-chloro-1-ethyl-1H-pyrrolo[2,3-c]pyridin-3-yl)-6-methylpyridin-4-yl]ethyl}amino)pyridine-2-carboxylic acid;
3-({1-[6-(5-chloro-1-methyl-1H-pyrrolo[2,3-c]pyridin-3-yl)-4-methylpyridin-2-yl]ethyl}amino)pyridine-2-carboxylic acid;
4-({(1R*)-1-[2-(5-chloro-1-methyl-1H-pyrrolo[2,3-c]pyridin-3-yl)-6-methylpyridin-4-yl]ethyl}amino)-1-(difluoromethyl)-1H-pyrazole-3-carboxylic acid;
4-({(1S*)-1-[2-(5-chloro-1-methyl-1H-pyrrolo[2,3-c]pyridin-3-yl)-6-methylpyridin-4-yl]ethyl}amino)-1-(difluoromethyl)-1H-pyrazole-3-carboxylic acid;
3-({1-[2-(5-chloro-1-methyl-1H-pyrrolo[2,3-c]pyridin-3-yl)-6-methylpyrimidin-4-yl]ethyl}amino)pyridine-2-carboxylic acid;
3-({(1R)-1-[3-methyl-5-(1-methyl-1H-pyrazolo[3,4-c]pyridin-3-yl)phenyl]ethyl}amino)pyridine-2-carboxylic acid;
3-({(1R)-1-[2-(5-chloro-1-methyl-1H-pyrrolo[2,3-c]pyridin-3-yl)-6-methylpyridin-4-yl]ethyl}amino)-5-fluoropyridine-2-carboxylic acid;
3-({1-[2-(1H-1,3-benzimidazol-4-yl)-6-methylpyridin-4-yl]ethyl}amino)pyridine-2-carboxylic acid;
3-({(1R)-1-[2-(imidazo[1,2-a]pyridin-5-yl)-6-methylpyridin-4-yl]ethyl}amino)pyridine-2-carboxylic acid;
4-({(1S)-1-[2-(5-chloro-1-methyl-1H-pyrrolo[2,3-c]pyridin-3-yl)-6-methylpyridin-4-yl]ethyl}amino)-1,2-thiazole-3-carboxylic acid;
2-({(1S*)-1-[3-methyl-5-(5-methyl-5H-pyrrolo[3,2-d]pyrimidin-7-yl)phenyl]ethyl}amino)benzoic acid;
2-({(1R*)-1-[3-methyl-5-(5-methyl-5H-pyrrolo[3,2-d]pyrimidin-7-yl)phenyl]ethyl}amino)benzoic acid;
3-[(1-{2-[5-chloro-1-(propan-2-yl)-1H-pyrrolo[2,3-c]pyridin-3-yl]-6-methylpyridin-4-yl}ethyl)amino]pyridine-2-carboxylic acid;
2-({(1S*)-1-[3-(1,5-dimethyl-1H-pyrrolo[2,3-c]pyridin-3-yl)-5-methylphenyl]ethyl}amino)benzoic acid;
2-({(1R*)-1-[3-(1,5-dimethyl-1H-pyrrolo[2,3-c]pyridin-3-yl)-5-methylphenyl]ethyl}amino)benzoic acid;
3-({1-[2-(5-chloro-1-cyclopentyl-1H-pyrrolo[2,3-c]pyridin-3-yl)-6-methylpyridin-4-yl]ethyl}amino)pyridine-2-carboxylic acid;
2-fluoro-6-({(1R)-1-[3-methyl-5-(1-methyl-1H-pyrrolo[2,3-c]pyridin-3-yl)phenyl]ethyl}amino)benzoic acid;
3-({(1R*)-1-[3-methyl-5-(1-methyl-1H-pyrrolo[2,3-c]pyridin-3-yl)phenyl]ethyl}amino)pyridine-2-carboxamide; and
3-({(1S*)-1-[3-methyl-5-(1-methyl-1H-pyrrolo[2,3-c]pyridin-3-yl)phenyl]ethyl}amino)pyridine-2-carboxamide;
   or a pharmaceutically acceptable salt thereof.
   E42 A compound of embodiment E1 selected from the group consisting of:
3-({(1R*)-1-[2-(5-chloro-1-methyl-1H-pyrrolo[2,3-c]pyridin-3-yl)-6-methylpyridin-4-yl]ethyl}amino)pyridine-2-carboxylic acid;
2-({(1R*)-1-[2-methyl-6-(1-methyl-1H-pyrrolo[2,3-c]pyridin-3-yl) pyridin-4-yl]ethyl}amino)benzoic acid;
2-({(1R*)-1-[3-methyl-5-(1-methyl-1H-pyrrolo[2,3-c]pyridin-3-yl)phenyl]ethyl}amino)benzoic acid;
3-({(1R*)-1-[3-methyl-5-(1-methyl-1H-pyrrolo[2,3-c]pyridin-3-yl)phenyl]ethyl}amino)pyridine-2-carboxylic acid;
6-chloro-3-({(1R*)-1-[3-methyl-5-(1-methyl-1H-pyrrolo[2,3-c]pyridin-3-yl)phenyl]ethyl}amino)pyridine-2-carboxylic acid;
3-({(1R″)-1-[2-methyl-6-(1-methyl-1H-pyrrolo[2,3-c]pyridin-3-yl) pyridin-4-yl]ethyl}amino)pyridine-2-carboxylic acid;
4-({1-[2-(5-chloro-1-methyl-1H-pyrrolo[2,3-c]pyridin-3-yl)-6-methylpyridin-4-yl]ethyl}amino)-1-methyl-1H-pyrazole-3-carboxylic acid;
3-({(1R)-1-[2-(5-chloro-1-methyl-1H-pyrrolo[2,3-c]pyridin-3-yl)-6-methylpyridin-4-yl]ethyl}amino)-6-methylpyridine-2-carboxylic acid;
2-({(1R*)-1-[3-(5-methoxy-1-methyl-1H-pyrrolo[2,3-c]pyridin-3-yl)-5-methylphenyl]ethyl}amino)benzoic acid;
N-(methanesulfonyl)-2-({(1R)-1-[3-methyl-5-(1-methyl-1H-pyrrolo[2,3-c]pyridin-3-yl)phenyl]ethyl}amino)benzamide;
2-({(1R)-1-[2-(5-chloro-1-methyl-1H-pyrrolo[2,3-c]pyridin-3-yl)-6-methylpyridin-4-yl]ethyl}amino)-5-fluorobenzoic acid;
3-({[2-(5-chloro-1-methyl-1H-pyrrolo[2,3-c]pyridin-3-yl)-6-methylpyridin-4-yl]methyl}amino)pyridine-2-carboxylic acid;
3-({(1R)-1-[2-(5-chloro-1-methyl-1H-pyrrolo[2,3-c]pyridin-3-yl)-6-methylpyridin-4-yl]ethyl}amino)pyridine-2-carboxamide;
4-({1-[2-(5-chloro-1-methyl-1H-pyrrolo[2,3-c]pyridin-3-yl)-6-methylpyridin-4-yl]ethyl}amino)-1-methyl-1H-pyrazole-5-carboxylic acid;
3-({1-[2-(5-chloro-1-ethyl-1H-pyrrolo[2,3-c]pyridin-3-yl)-6-methylpyridin-4-yl]ethyl}amino)pyridine-2-carboxylic acid;
3-({1-[6-(5-chloro-1-methyl-1H-pyrrolo[2,3-c]pyridin-3-yl)-4-methylpyridin-2-yl]ethyl}amino)pyridine-2-carboxylic acid;
4-({(1R*)-1-[2-(5-chloro-1-methyl-1H-pyrrolo[2,3-c]pyridin-3-yl)-6-methylpyridin-4-yl]ethyl}amino)-1-(difluoromethyl)-1H-pyrazole-3-carboxylic acid;
3-({1-[2-(5-chloro-1-methyl-1H-pyrrolo[2,3-c]pyridin-3-yl)-6-methylpyrimidin-4-yl]ethyl}amino)pyridine-2-carboxylic acid;
3-({(1R)-1-[3-methyl-5-(1-methyl-1H-pyrazolo[3,4-c]pyridin-3-yl)phenyl]ethyl}amino)pyridine-2-carboxylic acid;
3-({(1R)-1-[2-(5-chloro-1-methyl-1H-pyrrolo[2,3-c]pyridin-3-yl)-6-methylpyridin-4-yl]ethyl}amino)-5-fluoropyridine-2-carboxylic acid;
3-({1-[2-(1H-1,3-benzimidazol-4-yl)-6-methylpyridin-4-yl]ethyl}amino)pyridine-2-carboxylic acid;
3-({(1R)-1-[2-(imidazo[1,2-a]pyridin-5-yl)-6-methylpyridin-4-yl]ethyl}amino)pyridine-2-carboxylic acid;
4-({(1S)-1-[2-(5-chloro-1-methyl-1H-pyrrolo[2,3-c]pyridin-3-yl)-6-methylpyridin-4-yl]ethyl}amino)-1,2-thiazole-3-carboxylic acid;
2-({(1S*)-1-[3-methyl-5-(5-methyl-5H-pyrrolo[3,2-d]pyrimidin-7-yl)phenyl]ethyl}amino)benzoic acid;
3-[(1-{2-[5-chloro-1-(propan-2-yl)-1H-pyrrolo[2,3-c]pyridin-3-yl]-6-methylpyridin-4-yl}ethyl)amino]pyridine-2-carboxylic acid;
2-({(1S*)-1-[3-(1,5-dimethyl-1H-pyrrolo[2,3-c]pyridin-3-yl)-5-methylphenyl]ethyl}amino)benzoic acid;
3-({1-[2-(5-chloro-1-cyclopentyl-1H-pyrrolo[2,3-c]pyridin-3-yl)-6-methylpyridin-4-yl]ethyl}amino)pyridine-2-carboxylic acid;
2-fluoro-6-({(1R)-1-[3-methyl-5-(1-methyl-1H-pyrrolo[2,3-c]pyridin-3-yl)phenyl]ethyl}amino)benzoic acid; and
3-({(1R*)-1-[3-methyl-5-(1-methyl-1H-pyrrolo[2,3-c]pyridin-3-yl)phenyl]ethyl}amino)pyridine-2-carboxamide;
   or a pharmaceutically acceptable salt thereof.

Any of the compounds described in embodiments E40 to E42, or pharmaceutically acceptable salts thereof, may be claimed individually or grouped together with one or more other compounds of embodiments E1 to E39, or pharmaceutically acceptable salts thereof.
E43 A compound of any one of embodiments E1 to E42, or a pharmaceutically acceptable salt thereof, wherein one or more hydrogen atoms of the compound are replaced with deuterium.
E44 A compound of any one of embodiments E1 to E43, or a pharmaceutically acceptable salt thereof, wherein one, two, or three hydrogen atoms of the compound is/are replaced with deuterium. In a further embodiment, three hydrogen atoms of the compound are replaced with deuterium.
E45 A pharmaceutical composition comprising a compound of any one of embodiments E1 to E44, or a pharmaceutically acceptable salt thereof, and at least one pharmaceutically acceptable excipient.
E46 A method for treating cancer, comprising administering to a subject in need thereof a therapeutically effective amount of a compound of any one of embodiments E1 to E44, or a pharmaceutically acceptable salt thereof.
E47 A method for treating cancer, comprising administering to a subject in need thereof a therapeutically effective amount of a compound of any one of embodiments E1 to E44, or a pharmaceutically acceptable salt thereof, as a single agent.
E48 A method for treating cancer, comprising administering to a subject in need thereof a therapeutically effective amount of a compound of any one of embodiments E1 to E44, or a pharmaceutically acceptable salt thereof, and further comprising administering a therapeutically effective amount of an additional anti-cancer therapeutic agent.
E49 A compound of any one of embodiments E1 to E44, or a pharmaceutically acceptable salt thereof, for use as a medicament.
E50 A compound of any one embodiments E1 to E44, or a pharmaceutically acceptable salt thereof, for use in the treatment of cancer.
E51 Use of a compound of any one of embodiments E1 to E44 or a pharmaceutically acceptable salt thereof for the manufacture of a medicament for the treatment of cancer.
E52 A method for the treatment of a disorder mediated by PI3K-α in a subject, comprising administering to the subject in need thereof a compound of any one of embodiments E1 to E44, or a pharmaceutically acceptable salt thereof, in an amount that is effective for treating the disorder.
E53 A pharmaceutical combination comprising a compound of any one of embodiments E1 to E44 or a pharmaceutically acceptable salt thereof, and at least one additional therapeutic agent or a pharmaceutically acceptable salt thereof.
E54 A pharmaceutical composition comprising the pharmaceutical combination of embodiment E53 and at least one excipient.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein.

Each of the Formulas that are within the scope of other Formulas are also envisioned for the Formula (e.g., embodiments of Formula (I) include embodiments of Formula (Ia), Formula (II), Formula (IIa), Formula (I′), Formula (Ia′), Formula (II′), and Formula (IIa′)). Furthermore, each of the embodiments described herein envisions within its scope pharmaceutically acceptable salts of the compounds described herein.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

The terms PI3K-α mut, PI3K-α mutant, PI3K-α pan mutant and PI3K-α pan mutant selective are used herein.

The terms “PI3K-α inhibitor,” “PI3Kα inhibitor,” “PI3K alpha inhibitor,” “alpha-isoform specific phosphatidylinositol 3-kinase inhibitor,” “alpha-isoform specific PI3K inhibitor,” “alpha-isoform selective phosphatidylinositol 3-kinase inhibitor,” and “alpha-isoform PI3K selective inhibitor” as used herein refer to a compound that selectively targets, modulates, decreases, or inhibits at least one activity of the alpha-isoform of PI3K (including wild type and mutant forms) with respect to beta and/or delta and/or gamma subtypes. Exemplary alpha-isoform specific PI3K inhibitors are disclosed in International PCT Application WO2010/029082, which is hereby incorporated by reference in its entirety.

The terms “comprise”, “comprising”, “include”, “including”, and “includes” when used in this specification and claims are intended to specify the presence of stated features, integers, components, or steps, but they do not preclude the presence or addition of one or more other features, integers, components, steps, or groups thereof.

“Compounds of the invention” include compounds of Formula (I) and the novel intermediates used in the preparation thereof. One of ordinary skill in the art will appreciate that compounds of the invention include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention include solvates, hydrates, isomorphs, polymorphs, esters, salt forms, prodrugs, and isotopically labelled versions thereof (including deuterium substitutions), where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5 mg±10%, i.e., it may vary between 4.5 mg and 5.5 mg.

If substituents are described as being “independently selected” from a group, each substituent is selected independent of the other. Each substituent therefore may be identical to or different from the other substituent(s).

A “bond” refers to a covalent linkage between two atoms, or two moieties when the atoms joined by the bond are considered to be part of larger substructure. In one aspect, when a group described herein is a bond, the referenced group is absent thereby allowing a bond to be formed between the remaining identified groups.

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I).

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Haloalkyl” refers to an alkyl group as defined above containing the specified number of carbon atoms wherein at least one hydrogen atom has been replaced by halogen. Haloalkyl groups man contain, but are not limited to, 1-6 carbon atoms (“C₁-C₆ haloalkyl”), 1-4 carbon atoms (“C₁-C₄ haloalkyl”), or 1-2 carbon atoms (“C₁-C₂ haloalkyl”). More specifically, fluorinated alkyl groups may be specifically referred to as “fluoroalkyl.”

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring. Cycloalkyl groups may contain, but are not limited to, 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

Illustrative examples of monocyclic heteroaryl groups include, but are not limited to a monovalent radical of:

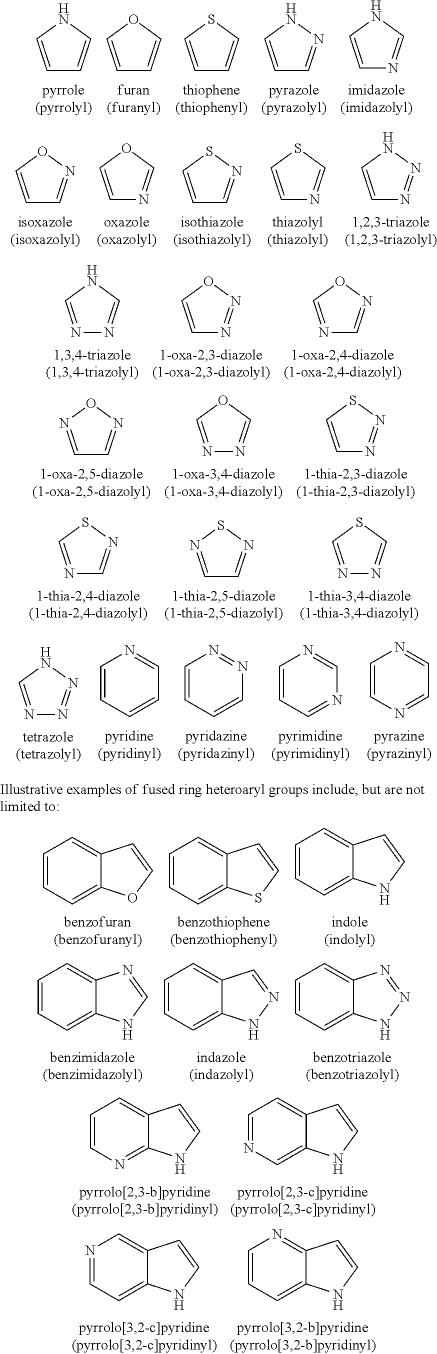

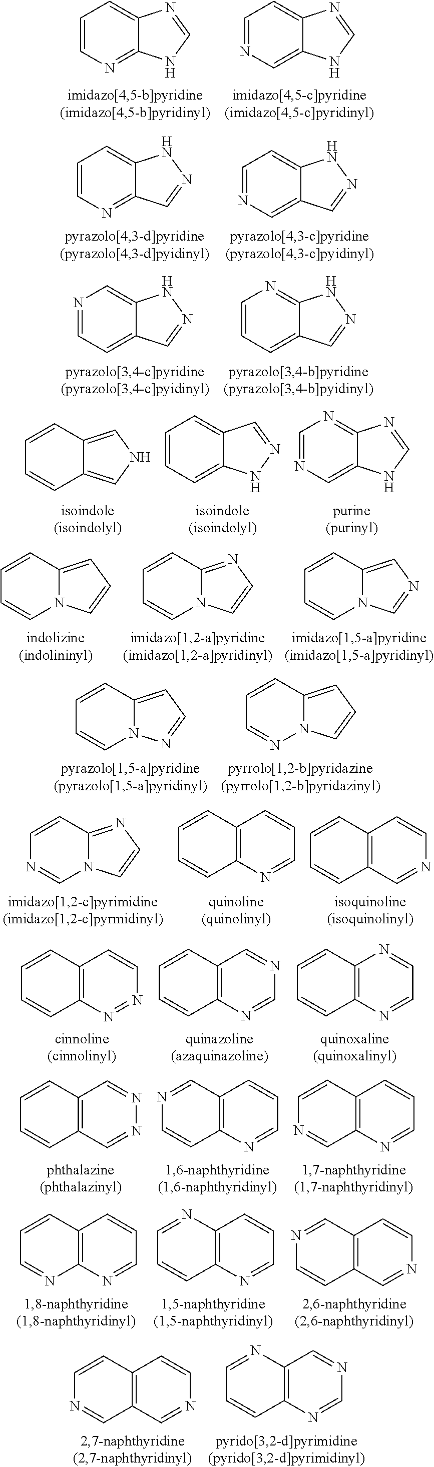

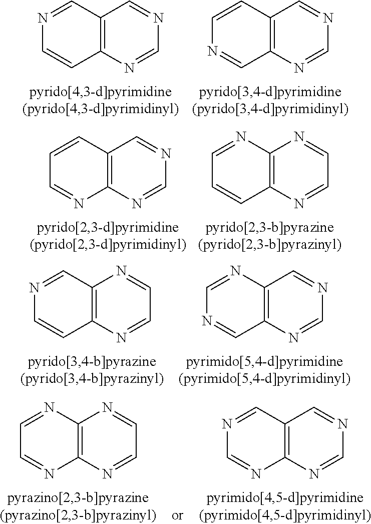

“Amino” refers to a group —NH₂, which is unsubstituted. Where the amino is described as substituted or optionally substituted, the term includes groups of the form —NR′R″, where each of R′ and R″ is defined as further described herein. For example, “alkylamino” refers to a group —NR′R″, wherein one of R′ and R″ is an alkyl moiety and the other is H, and “dialkylamino” refers to —NR′R″ wherein both of R′ and R″ are alkyl moieties, where the alkyl moieties have the specified number of carbon atoms (e.g., —NH(C₁-C₄ alkyl) or —N(C₁-C₄ alkyl)₂).

The term “pharmaceutically acceptable” means the substance (e.g., the compounds described herein) and any salt thereof, or composition containing the substance or salt of the invention is suitable for administration to a subject or patient.

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“Excipient” as used herein describes any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the term, “subject, “individual” or “patient,” used interchangeably, refers to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

The term “in vivo” refers to an event that takes place in a subject's body.

The term “in vitro” refers to an event that takes places outside of a subject's body. For example, an in vitro assay encompasses any assay conducted outside of a subject. In vitro assays encompass cell-based assays in which cells, alive or dead, are employed. In vitro assays also encompass a cell-free assay in which no intact cells are employed.

The term “combination,” “therapeutic combination,” or “pharmaceutical combination” as used herein refer to either a fixed combination in one dosage unit form, or non-fixed combination, or a kit of parts for the combined administration where two or more therapeutic agents may be administered independently, at the same time or separately within time intervals, especially where these time intervals allow that the combination partners show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

As used herein, the term “selective” describes a functionally-defined receptor ligand or enzyme inhibitor means selective for the defined receptor or enzyme subtype as compared with other receptor or enzyme subtypes in the same family. The term “selective inhibition” or “selectively inhibit” as applied to a biologically active agent refers to the agent's ability to selectively reduce the target signaling activity as compared to off-target signaling activity, via direct or indirect interaction with the target. For example, a compound that selectively inhibits one isoform of PI3K over another isoform of PI3K has an activity of at least greater than about 1× against a first isoform relative to the compound's activity against the second isoform (e.g., at least about 2×, 3×, 5×, 10×, 20×, 50×, 100×, 200×, 500×, or 1000×). In certain embodiments, these terms refer to (1) a compound described herein that selectively inhibits the gamma isoform over the alpha, beta, or delta isoform; or (2) a compound described herein that selectively inhibits the delta isoform over the alpha, beta, or gamma isoform. By way of non-limiting example, the ratio of selectivity can be greater than a factor of about 1, greater than a factor of about 2, greater than a factor of about 3, greater than a factor of about 5, greater than a factor of about 10, greater than a factor of about 50, greater than a factor of about 100, greater than a factor of about 200, greater than a factor of about 400, greater than a factor of about 600, greater than a factor of about 800, greater than a factor of about 1000, greater than a factor of about 1500, greater than a factor of about 2000, greater than a factor of about 5000, greater than a factor of about 10,000, or greater than a factor of about 20,000, where selectivity can be measured by IC₅₀. In certain embodiments, the IC₅₀ can be measured by in vitro or in vivo assays. In certain embodiments, the PI3K alpha isoform IC₅₀ activity of a compound provided herein can be less than about 1000 nM, less than about 500 nM, less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 5 nM, or less than about 1 nM.

In certain non-limiting embodiments, the compound of the invention that inhibits the PI3K/AKT pathway is an agent that selectively acts at the alpha isoform (p110a) of PI3K.

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formula (I) may also include other salts of such compounds which are not necessarily pharmaceutically acceptable salts, which may be useful as intermediates for one or more of the following: 1) preparing compounds of Formula (I); 2) purifying compounds of Formula (I); 3) separating enantiomers of compounds of Formula (I); or 4) separating diastereomers of compounds of Formula (I).

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekuhn, G. S., et al., “Trends in Active Pharmaceutical Ingredient Salt Selection Based on Analysis of the Orange Book Database,” J. Med. Chem. 50 (26) (2007): pp. 6665-6672.

Pharmaceutically acceptable salts of compounds of the invention may be prepared by methods well known to one skilled in the art, including but not limited to the following procedures
(i) by reacting a compound of the invention with the desired acid or base;
(ii) by removing an acid- or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates-see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together-see Chem Commun, 17; 1889-1896, by O. Almarsson and M. J. Zaworotko (2004). For a general review of multi-component complexes, see J Pharm Sci, 64 (8), 1269-1288, by Haleblian (August 1975).

### Solid Form

The compounds of the invention may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Hartshorne, N. H. and Stuart, A., Crystals and the Polarizing Microscope. 4^{th} Ed. London, Edward Arnold, 1970.

### Stereoisomers

Compounds of the invention may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the invention containing one or more asymmetric carbon atoms may exist as two or more stereoisomers. Where a compound of the invention contains an alkenyl or alkenylene group, geometric cis/trans (or Z/E) isomers are possible. Cis/trans isomers may also exist for saturated rings.

The compounds of the formulae provided herein may have asymmetric carbon atoms. The carbon-carbon bonds of the compounds of the disclosure may be depicted herein-using a solid line (-), a solid wedge ( [symbol] ), or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the disclosure may contain more than one asymmetric carbon atom. In those compounds, the use of a solid line to depict bonds to asymmetric carbon atoms is meant to indicate that all possible stereoisomers are meant to be included and the attached stereocenter. For example, unless stated otherwise, it is intended that the compounds of the disclosure can exist as enantiomers and diastereomers or as racemates and mixtures thereof. The use of a solid line to depict bonds to one or more asymmetric carbon atoms in a compound of the disclosure and the use of a solid or dotted wedge to depict bonds to other asymmetric carbon atoms in the same compound is meant to indicate that a mixture of diastereomers is present. In certain instances, the compounds may be prepared as a mixture of isomers such as a mixture of enantiomers or mixture of diastereomers and the mixture is then separated into the individual enantiomers or diastereomers. When the absolute stereochemistry is not known or not determined or not confirmed, the software-generated names are modified to include the symbol (R*) or (S*) indicating a single isomer with unknown stereochemistry, and the chemical structures are modified to include an asterisk “*” at the chiral center where stereochemistry is not known or not determined or not confirmed.

The pharmaceutically acceptable salts of compounds of the invention may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, R. M., Supercritical Fluid Chromatography with Packed Columns. 1^{st} Ed. RSC Chromatography Monographs, 1988.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art-see, for example, Eliel, E. L. and Wilen, S. H., Stereochemistry of Organic Compounds. 1^{st} Ed. New York, Wiley, 1994.

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, the deuterium compound is selected from any one of the compounds set forth in Deuterated Analog Tables shown in the Examples section.

In some embodiments, one or more hydrogen atoms on certain metabolic sites on the compounds of the invention are deuterated.

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

Unless otherwise stated, all isotopic substitution of the compounds of the invention are within the scope of the invention. Such compounds are useful, for example, as analytical tools, as probes in biological assays, or as therapeutic agents in accordance with the present invention.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., t-BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Metabolites

Also included within the scope of the invention are active metabolites of compounds of the invention, that is, compounds formed in vivo upon administration of the drug, often by oxidation or dealkylation. Some examples of metabolites in accordance with the invention include, but are not limited to:
(i) where the compound of the invention contains an alkyl group, a hydroxyalkyl derivative thereof (—CH>—COH):
(ii) where the compound of the invention contains an alkoxy group, a hydroxy derivative thereof (—OR->—OH);
(iii) where the compound of the invention contains a tertiary amino group, a secondary amino derivative thereof (—NRR′->—NHR or —NHR);
(iv) where the compound of the invention contains a secondary amino group, a primary derivative thereof (—NHR->—NH₂);
(v) where the compound of the invention contains a phenyl moiety, a phenol derivative thereof (-Ph->-PhOH);
(vi) where the compound of the invention contains an amide group, a carboxylic acid derivative thereof (—CONH₂->COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

The compositions of this invention may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the invention. In another embodiment, the oral administration may be in a powder or granule form. In another embodiment, the oral dosage form is sub-lingual, such as, for example, a lozenge. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the invention comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the invention comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated-see, for example, Finnin, B. C. and Morgan, T. M., “Transdermal penetration enhancers: Applications, limitations, and potential.” J. of Pharm. Sci. 1999, 88 (10): 955-958.

Formulations suitable for topical administration to the eye include, for example, eye drops wherein the compound of this invention is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the invention comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Allen, L. V. and Anesel, H. C. Ansel's Pharmaceutical Dosage Forms and Drug Delivery Systems. 10^{th} Ed. Philadelphia, Lippincott Williams & Wilkins, 2014; Adejare, A. Remington: The Science and Practice of Pharmacy. 23^{rd} Ed. Philadelphia, Lippincott Williams & Wilkins, 2000; Rowe, R. C., et al. Handbook of Pharmaceutical Excipients. 5^{th} Ed. Chicago, Pharmaceutical Press, 2006; Stahl, P. H. and Wermuth, C. G., Pharmaceutical Salts: Properties, Selection, and Use. 2^{nd} Revised Ed. New York, Wiley-VCH, 2011; and Brittain, H. G. Polymorphism in Pharmaceutical Solids. 2^{nd} Ed. CRC Press, 2009.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenously doses may range from about 0.01 to about 10 mg/kg/minute during a constant rate infusion.

Liposome containing compounds of the invention may be prepared by methods known in the art (See, for example, Chang, H. I. and Yeh, M. K., “Clinical development of liposome-based drugs: formulation, characterization, and therapeutic efficacy,” Int. J. of Nanomedicine. 2012, 7:49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Adejare, A. Remington: The Science and Practice of Pharmacy. 23^{rd} Ed. Philadelphia, Lippincott Williams & Wilkins, 2000.

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the invention are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the invention with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device, or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid-state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, Shah, N., et al. Amorphous Solid Dispersions: Theory and Practice. New York, Springer, 2014.

### Administration and Dosing

Typically, a compound of the invention is administered in an amount effective to treat a condition as described herein. The compounds of the invention may be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended. The compounds of the invention may be administered orally, rectally, vaginally, parenterally, topically, intranasally, or by inhalation.

The compounds of the invention may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the compounds of the invention may also be administered topically to the skin or mucosa, that is, dermally or transdermally. In another embodiment, the compounds of the invention may also be administered intranasally or by inhalation. In another embodiment, the compounds of the invention may be administered rectally or vaginally. In another embodiment, the compounds of the invention may also be administered directly to the eye or ear.

The dosage regimen for the compounds of the invention or compositions containing said compounds is based on a variety of factors, including the type, age, body, weight, sex and medical condition of the patient; the severity of the condition; the route of administration; the activity of the particular compound employed, diet, time of administration, rate of excretion, drug combination, and the judgment of the treating physician and the severity of the particular disease being treated. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

In some embodiments of the invention, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range and may be combined with any other dosage to constitute a dosage range comprising an upper and lower limit. In some embodiments, pharmaceutically acceptable compositions contain a provided compound and/or a pharmaceutically acceptable salt thereof at a concentration ranging from about 0.01 to about 90 wt %, about 0.01 to about 80 wt %, about 0.01 to about 70 wt %, about 0.01 to about 60 wt %, about 0.01 to about 50 wt %, about 0.01 to about 40 wt %, about 0.01 to about 30 wt %, about 0.01 to about 20 wt %, about 0.01 to about 2.0 wt %, about 0.01 to about 1 wt %, about 0.05 to about 0.5 wt %, about 1 to about 30 wt %, or about 1 to about 20 wt %.

### Therapeutic Methods and Uses

Provided herein are methods for inhibiting PI3K-α, encoded by PIK3CA gene. For example, provided herein are inhibitors of PI3K-α useful for treating or preventing diseases or disorders associated with dysregulation of a PIK3CA gene, a PI3K-α protein, or the expression or activity or level of any of the same (i.e., a PI3K-α associated disease or disorder), such as PIK3CA-related overgrowth syndromes ((PROS), see, e.g., Venot, Q., et al. “Targeted therapy in patients with PIK3CA-related overgrowth syndrome.” Nature. 558:540-546 (2018)), brain disorders (e.g., as macrocephaly-capillary malformation (MCAP) and hemimegalencephaly), congenital lipomatous (e.g., overgrowth of vascular malformations), epidermal nevi and skeletal/spinal anomalies (e.g., CLOVES syndrome) and fibroadipose hyperplasia (FH), or cancer (e.g., PI3K-α associated cancer).

A “PI3K-α inhibitor” as used herein includes any compound exhibiting PI3K-α inactivation activity (e.g., inhibiting or decreasing). In some embodiments, a PI3K-α inhibitor can be selective for a PI3K-α having one or more mutations.

Compounds of Formula (I), or pharmaceutically acceptable salts thereof, are useful for treating diseases and disorders which can be treated with a PI3K-α inhibitor, such as PI3K-α associated diseases and disorders, e.g., PIK3CA-related overgrowth syndromes (PROS) and proliferative disorders such as cancers, including hematological cancers and solid tumors (e.g., advanced or metastatic solid tumors).

The term “PI3K-α associated disease or disorder” as used herein refers to diseases or disorders associated with or having a dysregulation of a PIK3CA gene, a PI3K-α protein, or the expression or activity or level of any (e.g., one or more) of the same (e.g., any of the types of dysregulation of a PIK3CA gene, or a PI3K-α protein, or the expression or activity or level of any of the same described herein). Non-limiting examples of a PI3K-α associated disease or disorder include, for example, PIK3CA-related overgrowth syndromes (“PROS”), brain disorders (e.g., as macrocephaly-capillary malformation (MCAP) and hemimegalencephaly), congenital lipomatous (e.g., overgrowth of vascular malformations), epidermal nevi and skeletal/spinal anomalies (e.g., CLOVES syndrome) and fibroadipose hyperplasia (FH), or cancer (e.g., PI3K-α associated cancer).

The term “PI3K-α associated cancer” as used herein refers to cancers associated with or having a dysregulation, deregulation and/or alteration of a PIK3CA gene, a PI3K-α protein, and/or expression or activity, or level of any of the same.

Provided herein is a method of treating cancer (e.g., a PI3K-α associated cancer) in a subject in need of such treatment, the method comprising administering to the subject a therapeutically effective amount of a compound of Formula (I), or a pharmaceutically acceptable salt thereof, or a pharmaceutical composition thereof. For example, provided herein are methods for treating PI3K-α associated cancer in a subject in need of such treatment, the method comprising a) detecting a dysregulation, deregulation and/or alteration of PIK3CA gene, a PI3K-α protein, and/or the expression or activity or level of any of the same in a sample from the subject; and b) administering a therapeutically effective amount of a compound of Formula (I), or a pharmaceutically acceptable salt thereof. In some embodiments, the dysregulation of a PIK3CA gene, a PI3K-α protein, or the expression or activity or level of any of the same includes one or more a PI3K-α protein substitutions/point mutations/insertions.

Examples of PI3K-α DNA/mRNA/protein mutation/substitution/insertion/deletion included but are not limited to E542A, E542G, E542K, E542Q, E542V, E545A, E545D, E545G, E545K, E545Q, M1043I, M1043L, M1043T, M1043V, H1047L, H1047Q, H1047R, H1047Y, G1049R, or combinations thereof. In some embodiments, the PI3K-α DNA/mRNA/protein mutation/substitution/insertion/deletion is H1047X, where X is any amino acid.

In some embodiments of any of the methods or uses described herein, the cancer (e.g., PI3K-α associated cancer) is selected from the group consisting of a hematological cancer and a solid tumor.

In some embodiments of any of the methods or uses described herein, the cancer (e.g., PI3K-α associated cancer) is selected from the group consisting of breast cancer (including both HER2+ and HER2″ breast cancer, ER+ breast cancer, and triple negative breast cancer), endometrial cancer, lung cancer (including adenocarcinoma lung cancer and squamous cell lung carcinoma), esophageal squamous cell carcinoma, ovarian cancer, colorectal cancer, esophagogastric adenocarcinoma, bladder cancer, head and neck cancer (including head and neck squamous cell cancers such as oropharyngeal squamous cell carcinoma), thyroid cancer, glioma, cervical cancer, lymphangioma, meningioma, melanoma (including uveal melanoma), kidney cancer, pancreatic neuroendocrine neoplasms (pNETs), stomach cancer, esophageal cancer, acute myeloid leukemia, relapsed and refractory multiple myeloma, and pancreatic cancer.

In some embodiments of any of the methods or uses described herein, the cancer (e.g., PI3K-α associated cancer) is selected from the group consisting of breast cancer (including both HER2+ and HER2″ breast cancer, ER+ breast cancer, and triple negative breast cancer), colon cancer, rectal cancer, colorectal cancer, ovarian cancer, lymphangioma, meningioma, head and neck squamous cell cancer (including oropharyngeal squamous cell carcinoma), melanoma (including uveal melanoma), kidney cancer, pNETs, stomach cancer, esophageal cancer, acute myeloid leukemia, relapsed and refractory multiple myeloma, pancreatic cancer, lung cancer (including adenocarcinoma lung cancer and squamous cell lung carcinoma), and endometrial cancer.

In some embodiments of any of the methods or uses described herein, the cancer (e.g., PI3K-α associated cancer) is selected from the group consisting of breast cancer, lung cancer, endometrial cancer, esophageal squamous cell carcinoma, ovarian cancer, colorectal cancer, esophagogastric adenocarcinoma, bladder cancer, head and neck cancer, thyroid cancer, glioma, and cervical cancer.

In some embodiments of any of the methods or uses described herein, the cancer (e.g., PI3K-α associated cancer) is selected from the group consisting of HR+/HER2− breast cancer, colorectal cancer, and bladder cancer. In some embodiments of any of the methods or uses described herein, the cancer is HR+/HER2− breast cancer. In some embodiments of any of the methods or uses described herein, the cancer is colorectal cancer. In some embodiments of any of the methods or uses described herein, the cancer is bladder cancer.

In some embodiments of any of the methods or uses described herein, the PI3K-α associated cancer is breast cancer. In some embodiments of any of the methods or uses described herein, the PI3K-α associated cancer is colorectal cancer. In some embodiments of any of the methods or uses described herein, the PI3K-α associated cancer is endometrial cancer. In some embodiments of any of the methods or uses described herein, the PI3K-α associated cancer is lung cancer.

In some embodiments, compounds of Formula (I), or pharmaceutically acceptable thereof, are useful for treating a cancer that has been identified as having one or more PI3K-α mutations. Accordingly, provided herein are methods for treating a subject diagnosed with (or identified as having) a cancer that include administering to the subject a therapeutically effective amount of a compound of Formula (I), or a pharmaceutically acceptable salt thereof.

Also provided herein are methods for treating a subject identified or diagnosed as having a PI3K-α associated cancer that include administering to the subject a therapeutically effective amount of a compound of Formula (I), or a pharmaceutically acceptable salt thereof, or a pharmaceutical composition thereof. In some embodiments, the subject that has been identified or diagnosed as having a PI3K-α associated cancer through the use of a regulatory agency-approved, e.g., FDA-approved test or assay for identifying dysregulation of PIK3CA gene, a PI3K-α protein, or expression or activity or level of any of the same, in a subject or a biopsy sample from the subject or by performing any of the non-limiting examples of assays described herein. In some embodiments, the test or assay is provided as a kit. In some embodiments, the cancer is an PI3K-α associated cancer.

### Co-Administration

The terms “co-administration of” and “co-administering” and their grammatical equivalents, as used herein, encompass administration of two or more agents to subject so that both agent and/or their metabolites are present in the subject at the same or substantially the same time. In one embodiment, co-administration of a PI3K inhibitor with an additional anti-cancer agent (both components referred to hereinafter as the “two active agents”) refer to any administration of the two active agents, either separately or together, where the two active agents are administered as part of an appropriate dose regimen designed to obtain the benefit of the combination therapy.

In certain embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), essentially concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

Accordingly, the compounds of the invention may be used alone, or in combination with one or more other therapeutic agents, including the pharmaceutically acceptable salts of the specifically named agents and the pharmaceutically acceptable solvates of said agents and salts. The invention provides any of the uses, methods or compositions as defined herein wherein the compound of the invention, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The terms “fixed combination”, “fixed dose”, and “single formulation” refer to a compound of the invention, or a pharmaceutically acceptable salt thereof, and the one or more therapeutic agents, are both administered to a subject simultaneously in a single composition or dosage. The terms “non-fixed combination”, “kit of parts”, and “separate formulations” mean that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In some embodiments, a subject has a cancer (e.g., a locally advanced or metastatic tumor) that is refractory or intolerant to standard therapy (e.g., administration of a chemotherapeutic agent, such as a multikinase inhibitor, immunotherapy, or radiation (e.g., radioactive iodine)). In some embodiments, a subject has a cancer (e.g., a locally advanced or metastatic tumor) that is refractory or intolerant to prior therapy (e.g., administration of a chemotherapeutic agent, such as a multi-kinase inhibitor, immunotherapy, or radiation (e.g., radioactive iodine)).

In some embodiments, a subject has a cancer (e.g., a locally advanced or metastatic tumor) that has no standard therapy. In some embodiments, a subject is PI3K-α inhibitor naive. For example, the subject is naive to treatment with a PI3K-α selective inhibitor. In some embodiments, a subject is not PI3K-α inhibitor naive. In some embodiments, a subject is kinase inhibitor naive. In some embodiments, a subject is not kinase inhibitor naive. In some embodiments, a subject has undergone prior therapy. For example, treatment with a multi-kinase inhibitor (MKI) or another PI3K inhibitor, such as buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), ATP-competitive PI3K/mTOR dual inhibitor (PF-04691502), apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, or GSK2636771.

In some embodiments of any the methods described herein, the compound of Formula (I) (or a pharmaceutically acceptable salt thereof) is administered in combination with a therapeutically effective amount of at least one additional therapeutic agent.

Non-limiting examples of additional therapeutic agents include: other PI3K-α-targeted therapeutic agents (i.e., other PI3K-α inhibitors), c-Met receptor tyrosine kinase inhibitors, EGFR inhibitors, HER2 inhibitors, RAS pathway targeted therapeutic agents (including mTOR inhibitors), ATP-competitive PI3K/mTOR dual inhibitor, CDK inhibitors (e.g., CDK4/6 inhibitors (CDK4/6i), CDK2 inhibitor (CDK2i), CDK4 inhibitor (CDK4i)), estrogen receptor antagonist (fulvestrant), KRAS inhibitor, KAT6 inhibior (KAT6i), PARP inhibitors, other kinase inhibitors (e.g., receptor tyrosine kinase-targeted therapeutic agents (e.g., Trk inhibitors or multi-kinase inhibitors)), farnesyl transferase inhibitors, signal transduction pathway inhibitors, aromatase inhibitors, selective estrogen receptor modulators or degraders (SERMs/SERDs), estrogen receptor pro-deg, checkpoint inhibitors, modulators of the apoptosis pathway (e.g., obataclax); cytotoxic chemotherapeutics, angiogenesis-targeted therapies, immune-targeted agents, including immunotherapy, and radiotherapy.

In some embodiments, the EGFR inhibitor is osimertinib (AZD9291, merelectinib, TAGRISSO™), erlotinib (TARCEVA®), gefitinib (IRESSA®), cetuximab (ERBITUX®), necitumumab (PORTRAZZA™, IMC-11F8), neratinib (HKI-272, NERLYNX®), lapatinib (TYKERBR), panitumumab (ABX-EGF, VECTIBIX®), vandetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIFR), dacomitinib (PF-00299804, PF-804, PF-299, PF-299804), avitinib (AC0010), AC0010MA EAI045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyryl shikonin, acetylshikonin, P,P-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, RO5083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is osimertinib, gefitinib, erlotinib, afatinib, lapatinib, neratinib, AZD-9291, CL-387785, CO-1686, or WZ4002.

Exemplary HER2 inhibitors include trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPROR), afatinib (e.g., GILOTRIFR), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

In some embodiments, the compound of Formula (I) is combined with another PI3K inhibitor. In some embodiments, another PI3K inhibitor is a pan-PI3K inhibitor. In some embodiments, the another PI3K inhibitor is buparlisib (BKM120), alpelisib (BYL719), WX-037, copanlisib (ALIQOPATM, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, or a combination thereof.

Also provided herein is a method of treating cancer, comprising administering to a subject in need thereof a pharmaceutical combination for treating cancer which comprises (a) a compound of Formula (I), or a pharmaceutically acceptable salt thereof, (b) an additional therapeutic agent, and (c) optionally at least one pharmaceutically acceptable carrier for simultaneous, separate or sequential use for the treatment of cancer, wherein the amounts of the compound of Formula (I), or a pharmaceutically acceptable salt thereof, and the additional therapeutic agent are together effective in treating the cancer.

In some embodiments, the additional therapeutic agent(s) includes any one of the above listed therapies or therapeutic agents which are standards of care in cancers wherein the cancer has a dysregulation, deregulation and/or alteration of a PIK3CA gene, a PI3K-α protein, or expression or activity, or level of any of the same.

These additional therapeutic agents may be administered with one or more doses of the compound of Formula (I), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art.

Also provided herein is (i) a pharmaceutical combination for treating a cancer in a subject in need thereof, which comprises (a) a compound of Formula (I), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (I), or pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the cancer is a PI3K-α associated cancer.

The term “pharmaceutical combination”, as used herein, refers to a pharmaceutical therapy resulting from the mixing or combining of more than one active ingredient and includes both fixed and non-fixed combinations of the active ingredients, or a kit of parts for the combined administration where two or more therapeutic agents may be administered independently, at the same time or separately within time intervals, especially where these time intervals allow that the combination partners show a cooperative, e.g., synergistic, effect. The term “fixed combination” means that a compound of Formula (I), or a pharmaceutically acceptable salt thereof, and at least one additional therapeutic agent (e.g., a chemotherapeutic agent), are both administered to a subject simultaneously in the form of a single composition or dosage. The term “non-fixed combination” means that a compound of Formula (I), or a pharmaceutically acceptable salt thereof, and at least one additional therapeutic agent (e.g., chemotherapeutic agent) are formulated as separate compositions or dosages such that they may be administered to a subject in need thereof simultaneously, concurrently or sequentially with variable intervening time limits, wherein such administration provides effective levels of the two or more compounds in the body of the subject. These also apply to cocktail therapies, e.g., the administration of three or more active ingredients. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

Accordingly, also provided herein is a method of treating a cancer, comprising administering to a subject in need thereof a pharmaceutical combination for treating cancer which comprises (a) a compound of Formula (I), or pharmaceutically acceptable salt thereof, and (b) an additional therapeutic agent, wherein the compound of Formula (I) and the additional therapeutic agent are administered simultaneously, separately or sequentially, wherein the amounts of the compound of Formula (I), or pharmaceutically acceptable salt thereof, and the additional therapeutic agent are together effective in treating the cancer. In some embodiments, the compound of Formula (I), or pharmaceutically acceptable salt thereof, and the additional therapeutic agent are administered simultaneously as separate dosages. In some embodiments, the compound of Formula (I), or pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I), or pharmaceutically acceptable salt thereof, and the additional therapeutic agent are administered simultaneously as a combined dosage.

These agents and compounds of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents described herein or known in the art.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see Smith, M. B., March's Advanced Organic Chemistry: Reactions, Mechanisms, and Structure. 8^{th} Ed. New Jersey, Wiley, 2019.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

Experiments were conducted with mechanical stirring under an inert atmosphere of nitrogen or argon gas at room temperature unless otherwise stated. Deoxygenation (degassing) of oxygen-sensitive experiments was conducted via inert gas sparge (1-5 minutes) or by repeated vacuum/purge procedures (2-3 X) with inert gas. Commercial solvents and reagents were generally used without further purification and dried over molecular sieves (generally Sure-Seal™ products from the Aldrich Chemical Company, Milwaukee, Wisconsin). Mass spectrometry data is reported from either liquid chromatography-mass spectrometry (LC-MS), atmospheric pressure chemical ionization (APCI), electrospray ionization (ESI) or liquid chromatography-Time of Flight (LC-TOF) methods. Chemical shifts for nuclear magnetic resonance (NMR) data are expressed in parts per million (ppm) referenced to residual peaks from the deuterated solvents employed.

¹H and ¹⁹F Nuclear Magnetic Resonance (NMR) spectra were recorded on Bruker XWIN-NMR (400 or 600 MHz) spectrometer. ¹H and ¹⁹F resonances are reported in parts per million (ppm) downfield from tetramethylsilane. ¹H NMR data are reported as multiplicity (e.g., s, singlet; d, doublet; t, triplet; q, quartet; quint, quintuplet; dd, doublet of doublets; dt, doublet of triplets; br s, broad singlet; m, multiplet). For spectra obtained in CDCl₃, (CD₃)₂SO (or DMSO-d₆), and CD₃OD, the residual protons (7.27, 2.50, and 3.31 ppm, respectively) were used as the internal reference. All observed coupling constants, J, are reported in Hertz (Hz). “δ” means chemical shift. Exchangeable protons are not always observed.

Optical rotations were determined on a Jasco P-2000 or a Rudolph Autopol IV polarimeter. All final compounds were purified to ≥95% purity, unless otherwise specified. When absolute stereochemistry is known, (R,S) labels are used. When absolute stereochemistry is not known, the software-generated names are modified to include the symbol (R*) indicating one single isomer with unknown stereochemistry, and the chemical structures are modified to include “*” at the chiral center where the stereochemistry is not known.

For syntheses referencing procedures in other Examples or Methods, reaction Protocol (length of reaction and temperature) may vary. In general, reactions were followed by thin layer chromatography, LC-MS or HPLC, and subjected to work-up when appropriate. Purifications may vary between experiments: in general, solvents and the solvent ratios used for eluents/gradients were chosen to provide appropriate retention times. Unless otherwise specified, reverse phase HPLC fractions were concentrated via lyophilization/freeze-drying. Intermediate and final compounds were stored at (0° C.) or room temperature in closed vials or flasks under nitrogen.

### ABBREVIATIONS

Boc is tert-butoxycarbonyl;
br is broad;
t-Bu is tert-butyl;
° C. is degrees Celsius;
CDCl₃ is deutero-chloroform;
δ is chemical shift;
d is doublet;
dd is doublet of doublets;
ddd is doublet of doublet of doublets;
dt is doublet of triplets;
DMSO is dimethyl sulfoxide;
DMSO-d₆ is deuterodimethylsulfoxide;
EDCl is 1-ethyl-3-(3-dimethylaminopropyl)carbodiimide;
ESI is electrospray ionization;
g is gram;
HCl is hydrochloric acid;
HPLC is high pressure liquid chromatography;
HOBt is 1-hydroxybenzotriazole hydrate;
hr(s) is hour(s);
L is liter;
LCMS is liquid chromatography mass spectrometry;
m is multiplet;
M is molar;
MeOH is methanol;
mg is milligram;
MHz is mega Hertz;
min(s) is minute(s);
mL is milliliter;
mmol is millimole;
mol is mole;
MS (m/z) is mass spectrum peak;
NMR is nuclear magnetic resonance;
Pd(dppf)Cl₂ is [1,1′-bis(diphenylphophino)ferrocene]dichloropalladium(II);
pH is power of hydrogen;
ppm is parts per million;
q is quartet;
rt is room temperature;
RT is retention time;
s is singlet;
SFC is supercritical fluid chromatography;
t is triplet;
THF is tetrahydrofuran;
μL is microliter;
μmol is micromole; and
Xantphos is 4,5-bis(diphenylphosphno)-9,9-dimethylxanthene

### GENERAL METHODS

The Schemes described below are intended to provide a general description of the methodology employed in the preparation of the compounds of the present invention. Some of the compounds of the present invention contain a chiral center. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

Unless stated otherwise, the variables in Schemes I and II have the same meanings as defined herein. Compounds at every step may be purified by standard techniques, such as column chromatography, crystallization, or reverse phase SFC or HPLC (either chiral or achiral).

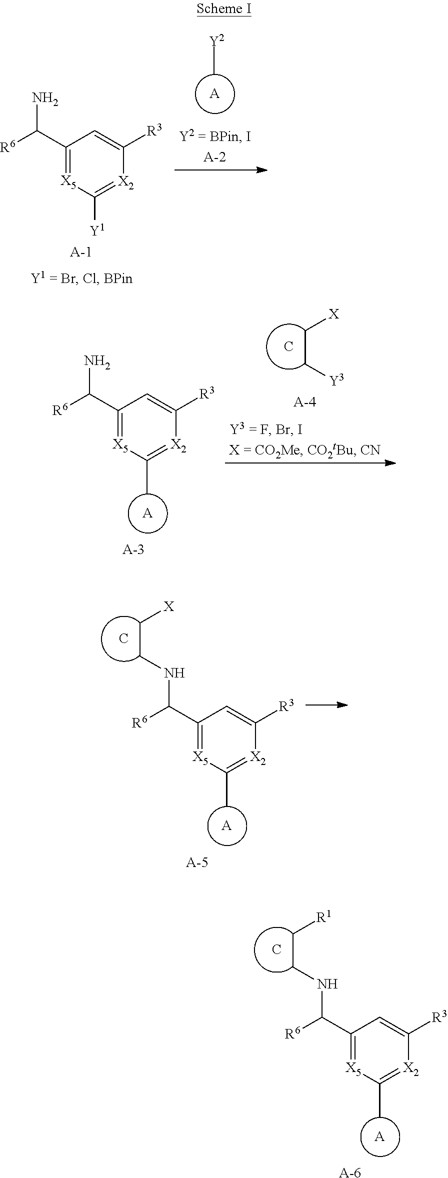

As exemplified in Scheme I, a compound such as A-3 can be accessed from a Suzuki coupling of compound A-1 (either purchased or synthesized) with compound A-2. Exposure of compounds such as A-3 and A-4 to nucleophilic aromatic substitution conditions or Buchwald-Hartwig amination conditions can deliver compounds such as A-5 which, in turn, can be saponified or hydrolyzed to deliver compounds such as A-6. In some cases, compound A-1 may have a protective group (e.g., S(O)t-Bu) on the amine functional group which can be cleaved following Suzuki coupling using conditions known in the art (Protective Groups in Organic Synthesis, A. Wiley-Interscience Publication, 1981; Protecting groups, 10 Georg Thieme Verlag, 1994; Smith, M. B., March's Advanced Organic Chemistry: Reactions, Mechanisms, and Structure. 8^{th} Ed. New Jersey, Wiley, 2019). In cases where A-2 has a protective group (e.g., Boc protected amine), it may be exchanged for an alkyl group prior to step 2 under standard conditions known in the art. In some cases, the carboxylic acid in compound A-6 may be further derivatized under standard conditions known in the art (e.g., acylsulfonamide formation). In some cases, A-3 may be functionalized (e.g., alkylated) under standard conditions known in the art.

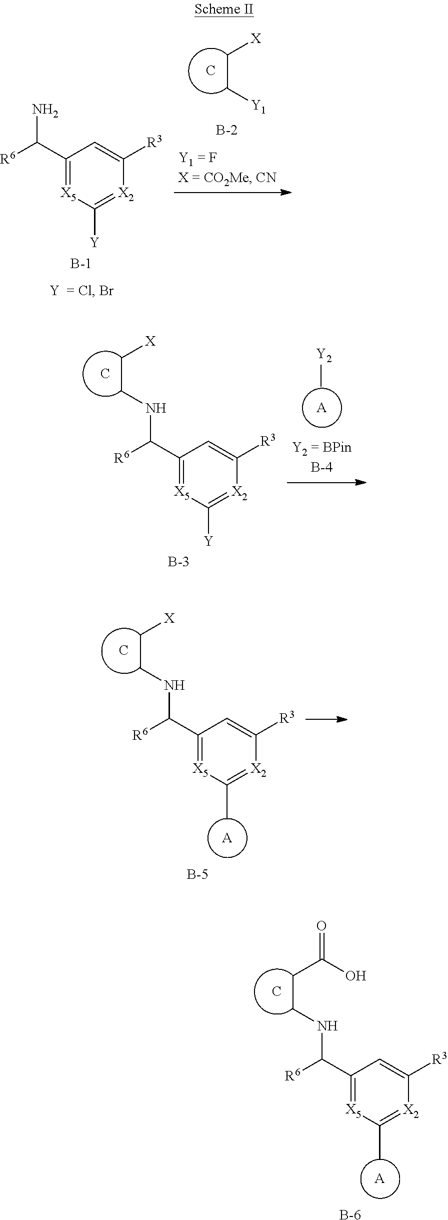

As exemplified in Scheme II, a compound such as B-3 can be synthesized from a compound such as B-1 (either purchased or synthesized) reacting with a compound such as B-2 under nucleophilic aromatic substitution conditions. Compound B-3 can, in turn, be coupled with compounds such as B-4 to deliver compounds such as B-5. Compounds such as B-6 can be synthesized by saponification or hydrolysis of compounds such as B-5. In some cases, the carboxylic acid in compound B-6 may be further derivatized under standard conditions known in the art (e.g., acylsulfonamide formation).

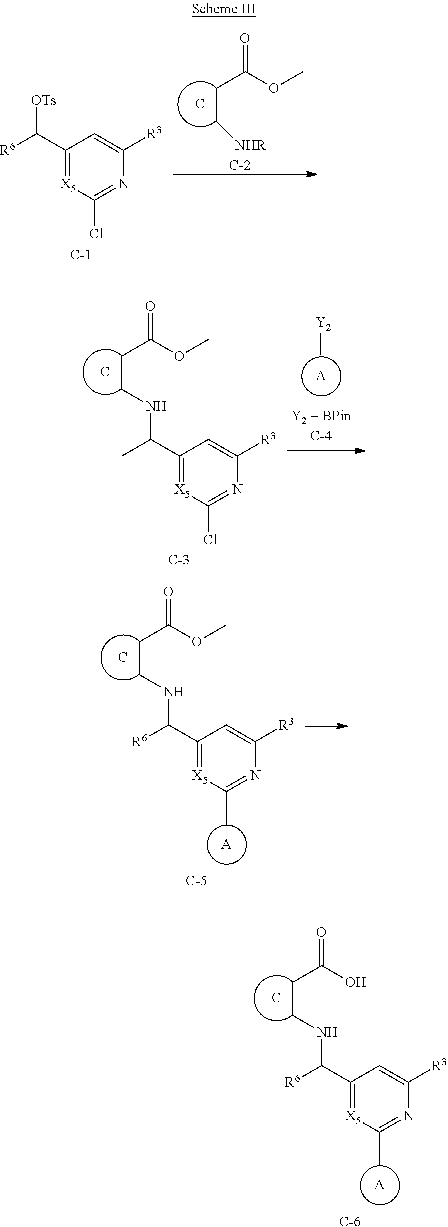

As exemplified in Scheme III, a compound such as C-3 can be obtained from a displacement reaction between a compound such as C-1 with an amine (appended to- or contained within a heterocyclic ring) such as C-2. Compounds such as C-5 can be obtained in turned by Suzuki coupling between compounds such as C-3 and C-4. Compounds such as C-6 can be synthesized by saponification or hydrolysis of compounds such as C-5.

### EXAMPLES

In order that this invention may be better understood, the following examples are set forth. These examples are for purposes of illustration only and are not to be construed as limiting the scope of the invention in any manner.

The compounds and intermediates described below were named using the naming convention provided with ACD/Spectrus Processor 2019.1.1, Version S05S41 (Advanced Chemistry Development, 8 King Street East, Suite 107, Toronto, Ontario, M5C 1B5, Canada). The naming convention provided with ACD/Spectrus Processor 2019.1.1, Version S05S41, is well known by those skilled in the art and it is believed that the naming convention provided with ACD/Spectrus Processor 2019.1.1, Version S05S41, generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

### INTERMEDIATES

### Example A

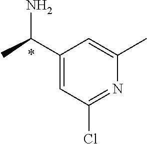

### (1R*)-1-(2-chloro-6-methylpyridin-4-yl)ethan-1-amine

Step 1: To a solution of 1-(2-chloro-6-methylpyridin-4-yl)ethan-1-one (32.0 g, 190 mmol) in THF (500 mL) was added (S)-(−)-2-methyl-2-propanesulfinaminde (2.58, 21.3 mmol) followed by titanium (IV) ethoxide (215 g, 943 mmol). The resultant mixture was heated to 60° C. for 16 hours. The mixture was then poured into ice-cold water (1.0 L), and the resultant mixture was extracted with ethyl acetate (3×500 mL). The combined extracts were washed with water (100 mL), dried over anhydrous sodium sulfate and concentrated. Silica gel chromatography eluting with ethyl acetate/petroleum ether afforded (S²S)—N-[(1Z)-1-(2-chloro-6-methylpyridin-4-yl)ethylidene]-2-methylpropane-2-sulfinamide (43 g, 84%) as an oil. LCMS m/z for C₁₂H₁₇ClN₂OS, 273.0 (M+H)⁺.

Step 2: To (S²S)—N-[(1Z)-1-(2-chloro-6-methylpyridin-4-yl)ethylidene]-2-methylpropane-2-sulfinamide (43 g, 160 mmol) in methanol (400 mL) at ice bath temperature was added sodium borohydride (7.93 g, 210 mmol). The mixture was allowed to warm to room temperature and was stirred for 2 hours. The reaction mixture was then quenched with water (50 mL) and concentrated under vacuum. The residue was diluted with water (100 mL) and extracted with ethyl acetate (3×100 mL). The combined extracts were washed with brine (50 mL), dried over anhydrous sodium sulfate, and concentrated. The residue was chromatographed over silica gel eluting with an ethyl acetate/petroleum ether gradient to afford the product diastereomers, each as solids. First to elute: (S²S)—N-[(1R*)-1-(2-chloro-6-methylpyridin-4-yl)ethyl]-2-methylpropane-2-sulfinamide (32.0 g, 74%); LCMS m/z for C₁₂H₁₉ClN₂OS, 275.0 (M+H)⁺. Second to elute: (S²S)—N-[(1S*)-1-(2-chloro-6-methylpyridin-4-yl)ethyl]-2-methylpropane-2-sulfinamide (5.7 g, 13%); LCMS m/z for C₁₂H₁₉ClN₂OS, 275.0 (M+H)⁺. Each sample was contaminated with significant quantities of the other diastereomer, so the lots were recombined and taken on to the next step.

Step 3: A mixture of (S²S)—N-[(1R*)-1-(2-chloro-6-methylpyridin-4-yl)ethyl]-2-methylpropane-2-sulfinamide and (S²S)—N-[(1S*)-1-(2-chloro-6-methylpyridin-4-yl)ethyl]-2-methylpropane-2-sulfinamide (37.5 g, 136 mmol) was dissolved in ethyl acetate (80.0 mL) with the aid of sonication for 10 minutes. The solution was concentrated under reduced pressure to an approximate volume of less than 30 mL. To the solution was then added hexane (100 mL), and the mixture was sonicated for 10 minutes. The resultant solid was collected via filtration to afford (S²S)—N-[(1R*)-1-(2-chloro-6-methylpyridin-4-yl)ethyl]-2-methylpropane-2-sulfinamide (15.9 g, 42%) was a solid. ¹H NMR (400 MHz, (CD₃)₂SO) δ 7.27 (d, J=17.5 Hz, 2H), 5.64-5.53 (m, 1H), 4.50-4.36 (m, 1H), 2.43 (s, 3H), 1.44 (d, J=6.8 Hz, 3H), 1.12 (s, 9H); LCMS m/z for C₁₂H₁₉ClN₂OS, 275.0 (M+H)⁺. The mother liquor was concentrated and chromatographed by preparative HPLC (column: 41-WePure Biotech XP tC18 150×40 mm, 7 μm; mobile phase: [A: 08-Water (0.05% NH₃H₂O+10 mM NH₄HCO₃); B: acetonitrile]; B %: 18.00%-58.00%, 12.00 min; flow rate: 60.00 mL/min). The product fractions were concentrated to remove acetonitrile, and the aqueous solution was then extracted with ethyl acetate (3×50 mL). The combined extracts were dried over sodium sulfate, filtered, and concentrated to afford a 1:1 mixture of both (R*) and (S*) isomers (16.0 g, 43%) as a gum. ¹H NMR (400 MHz, (CD₃)₂SO) δ 7.37 (s, 1H), 7.33-7.27 (m, 1H), 7.27-7.22 (m, 1H), 5.94-5.82 (m, 1H), 5.62-5.52 (m, 1H), 4.49-4.40 (m, 1H), 4.40-4.31 (m, 1H), 2.43 (d, J=2.4 Hz, 3H), 1.44 (d, J=6.8 Hz, 2H), 1.39-1.33 (m, 1H), 1.12 (s, 9H); LCMS m/z for C₁₂H₁₉ClN₂OS, 275.1 (M+H)⁺.

Step 4: To a mixture of (S²S)—N-[(1R*)-1-(2-chloro-6-methylpyridin-4-yl)ethyl]-2-methylpropane-2-sulfinamide (15.9 g, 57.9 mmol) in dichloromethane (50.0 mL) was added a solution of 2.0M HCl in dioxane (30 mL), and the mixture was stirred at room temperature for 2 hours. The mixture was then concentrated under vacuum, and the residue was crystallized from ethyl acetate/hexane (5:1) to afford (1R*)-1-(2-chloro-6-methylpyridin-4-yl)ethan-1-amine (12.5 g, 99%) as a solid. ¹H NMR (400 MHz, (CD₃)₂SO) δ 8.99-8.83 (m, 3H), 7.56 (s, 1H), 7.47 (s, 1H), 6.00-5.62 (m, 2H), 4.40 (td, J=5.8, 12.0 Hz, 1H), 2.45 (s, 3H), 1.50 (d, J=6.8 Hz, 3H). LCMS m/z for C₈H₁₁ClN₂, 171.1 (M+H)⁺; [α]=+27.6 (c 0.1, MeOH).

### Example B

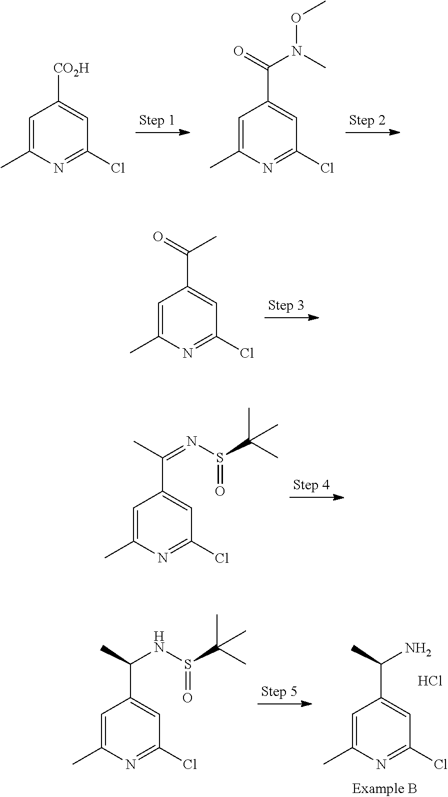

### (1R)-1-(2-chloro-6-methylpyridin-4-yl)ethan-1-amine hydrochloride

Step 1: To a solution of 2-chloro-6-methylpyridine-4-carboxylic acid (300 g, 1.75 mol) and N,O-dimethylhydroxylamine hydrochloride (205 g, 2.10 mol) in dichloromethane (1.50 L) at room temperature was added sequentially N,N-diisopropylethylamine (452 g, 3.50 mol), HOBt (284 g, 2.10 mol), and EDCl (402 g, 2.10 mol), and the resultant mixture was stirred for 2 h. Upon completion, the mixture was diluted with saturated sodium carbonate (2.0 L) and extracted with dichloromethane (2.0 L). The combined extracts were washed with saturated sodium carbonate (2×2.0 L), dried over anhydrous magnesium sulfate, filtered, and concentrated. The process was repeated for a total of eleven batches, which were combined to afford 2-chloro-N-methoxy-N,6-dimethylpyridine-4-carboxamide (3836 g, crude) as an oil, which was used in the next step without further treatment. ¹H NMR (400 MHz, (CD₃)₂SO) δ 7.42 (d, J=16.3 Hz, 2H), 3.57 (s, 3H), 3.27 (s, 3H), 2.53-2.50 (m, 3H); LCMS m/z for C₉H₁₁ClN₂O₂, 215.2 [M+H]⁺.

Step 2: This experiment was conducted at 0° C. in a flow reactor (PFA Coils reactor, 6.35 mm (¼″), 60 mL, residence time 1.0 min). Solution A, consisting of 2-chloro-N-methoxy-N,6-dimethylpyridine-4-carboxamide (1.50 kg, 6.99 mol) in THF (3.00 L), was introduced to the reactor via Pump A at a flow rate of 8.5 mL/min. Solution B, consisting of a solution of methylmagnesium bromide in THF (3.0M, 4.89 L, 14.7 mol), was concomitantly introduced to the reactor via Pump B at a flow rate of 8.5 mL/min. The product mixture was collected into a solution of 10% aqueous ammonium chloride (30.0 L) at ice bath temperature. Upon completion, the mixture was extracted with ethyl acetate (2×20 L), and the combined extracts were washed with water (2×20 L), dried over anhydrous magnesium sulfate, filtered, and concentrated to afford 1-(2-chloro-6-methylpyridin-4-yl)ethan-1-one (1.10 kg, 93%, crude) as an oil. The material was taken directly into the next step. ¹H NMR (400 MHz, (CD₃)₂SO) δ 7.69 (d, J=4.0 Hz, 2H), 2.61 (s, 3H), 2.54 (s, 3H); LCMS m/z for C₈H₈ClNO, 170.2 [M+H]⁺.

Step 3: To a solution of 1-(2-chloro-6-methylpyridin-4-yl)ethan-1-one (300 g, 1.77 mol) and (R)-2-methylpropane-2-sulfinamide (257 g, 2.12 mol) in THF (1.50 L) at room temperature was added titanium ethoxide (605 g, 2.65 mol), and the resultant mixture was then heated to 65° C. for 16 h. Upon completion, the mixture was diluted with water (2.0 L) and filtered. The filter cake was washed with ethyl acetate (2×500 mL), and the filtrate was extracted with ethyl acetate (2×2.0 L). The combined extracts were dried over anhydrous magnesium sulfate, filtered, and concentrated. The residue was purified by silica chromatography and eluted with hexane/ethyl acetate (3:1). The product fractions were collected and concentrated under reduced pressure, and the resultant residue was the triturated with hexanes (700 mL) for 1 h. The solids were collected via filtration and dried to afford (S²R)—N-[(1Z)-1-(2-chloro-6-methylpyridin-4-yl)ethylidene]-2-methylpropane-2-sulfinamide (233 g, 46%) as a solid. ¹H NMR (400 MHz, (CD₃)₂SO) δ 7.62 (br d, J=13.5 Hz, 2H), 2.71 (s, 3H), 2.52 (s, 3H), 1.22 (s, 9H); LCMS m/z for C₁₂H₁₇ClN₂OS, 273.2 [M+H]⁺.

Step 4: This experiment was conducted at room temperature in a flow reactor (FLR1, PFA Coils reactor, 6.35 mm (¼″), 800 mL, residence time 30 min). Solution A: (S²R)—N-[(1Z)-1-(2-chloro-6-methylpyridin-4-yl)ethylidene]-2-methylpropane-2-sulfinamide (400 g, 1.47 mol) in THF (2.00 L); Solution B: lithium tri-tert-butoxyaluminum hydride (4.00 L, 1.0 M in THF). Solution A was introduced via Pump 1 at a rate of 10 mL/min, and solution B was concomitantly introduced via Pump B at a rate of 20 mL/min. The product mixture was collected into a stirred volume of water (8.0 L) at ice bath temperature. The mixture was then extracted with ethyl acetate (2×4 L), and the combined extracts were dried over anhydrous magnesium sulfate, filtered, and concentrated under reduced pressure. The residue was dissolved in isopropyl ether and heated to 70° C. for 30 min. The resultant solution was then cooled to room temperature and seed crystals (4.0 g) were added followed by stirring 2 h. The resultant precipitate was then collected via filtration and dried under vacuum to afford (S²R)—N-[(1R)-1-(2-chloro-6-methylpyridin-4-yl)ethyl]-2-methylpropane-2-sulfinamide (302 g, 74%) as a solid. ¹H NMR (400 MHz, (CD₃)₂SO) δ 7.37 (s, 1H), 7.29 (s, 1H), 5.89 (d, J=8.4 Hz, 1H), 4.36 (quin, J=7.2 Hz, 1H), 2.43 (s, 3H), 1.37 (d, J=6.9 Hz, 3H), 1.12 (s, 9H); LCMS m/z for C₁₂H₁₉ClN₂OS, 275.2 [M+H]⁺.

Step 5: To (S²R)—N-[(1R)-1-(2-chloro-6-methylpyridin-4-yl)ethyl]-2-methylpropane-2-sulfinamide (235 g, 855 mmol) in ethyl acetate (2.50 L) at room temperature was added dropwise a solution of hydrochloric acid (1.17 L, 4.0M in ethyl acetate), and the resultant mixture was stirred for 2 h. The precipitate was then collected via filtration and dried under vacuum to afford (1R)-1-(2-chloro-6-methylpyridin-4-yl)ethan-1-amine hydrochloride (190 g, 96%) as a solid. ¹H NMR (400 MHz, (CD₃)₂SO) δ 9.79-9.21 (m, 1H), 9.09-8.68 (m, 3H), 7.61-7.52 (m, 1H), 7.51-7.44 (m, 1H), 4.48-4.34 (m, 1H), 2.49-2.42 (m, 3H), 1.53-1.48 (m, 3H); LCMS m/z for C₈H₁₂Cl₂N₂·HCl, 171.2 [M−Cl]⁺.

Additional intermediates shown below (Table A) were synthesized by modification of the methods exemplified above with non-critical changes or substitutions to the exemplified procedures that someone who is skilled in the art would be able to realize.

TABLE A

| Example | | |
|---|---|---|
| # | Structure/Compound Name | Characterizing Data |
| C | 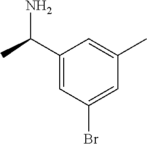 | ¹H NMR (400 MHz, DMSO-d₆) δ 8.61- 8.32 (m, 3H), 7.55 (s, 1H), 7.43 (s, 1H), 7.33 (s, 1H), 4.43-4.26 (m, 1H), 2.32 (s, 3H), 1.48 (d, J = 6.8 Hz, 3H); LCMS m/z (ESI) for C₉H₁₂BrN, 214.0 [M + H)⁺. |
| | (1R)-1-(3-bromo-5- | |
| | methylphenyl)ethan-1-amine | |
| D | 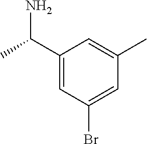 | ¹H NMR (400 MHz, DMSO-d₆) δ = 8.49 (br s, 3H), 7.55 (s, 1H), 7.43 (s, 1H), 7.34 (s, 1H), 4.36 (q, J = 6.7 Hz, 1H), 2.32 (s, 3H), 1.48 (d, J = 6.8 Hz, 3H); LCMS m/z (ESI) for C₉H₁₂BrN, 213.9 [M + H)⁺. |
| | (1S)-1-(3-bromo-5- | |
| | methylphenyl)ethan-1-amine | |
| E | 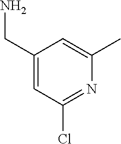 | ¹H NMR (400 MHz, DMSO-d₆) 8.67 (br s, 3H), 7.50 (s, 1H), 7.40 (s, 1H), 4.05 (q, J = 5.8 Hz, 2H), 2.46 (s, 3H); LCMS m/z (ESI) for C₇H₉ClN₂, 157.1 [M + H)⁺. |
| | 1-(2-chloro-6-methylpyridin-4- | |
| | yl)methanamine | |
| F | 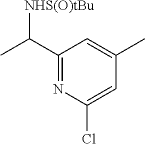 | LCMS m/z (ESI) for C₁₂H₁₉ClN₂OS, 273.1 [M + H]+ |
| | N-[1-(6-chloro-4-methylpyridin-2- | |
| | yl)ethyl]-2-methylpropane-2- | |
| | sulfinamide | |

### Example G

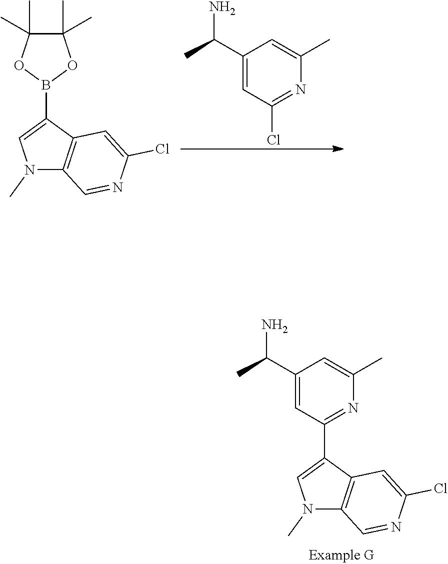 [SMILES: Cn1cc(B2OC(C)(C)C(C)(C)O2)c2cc(Cl)ncc21 ; Cc1cc([C@@H](C)N)cc(Cl)n1 ; Cc1cc([C@@H](C)N)cc(-c2cn(C)c3cnc(Cl)cc23)n1]

### (1R)-1-[2-(5-chloro-1-methyl-1H-pyrrolo[2,3-c]pyridin-3-yl)-6-methylpyridin-4-yl]ethan-1-amine

A mixture of 5-chloro-1-methyl-3-(4,4,5,5-tetramethyl-1,3,2-dioxaborolan-2-yl)-1H-pyrrolo[2,3-c]pyridine (135 g, 460 mmol), (1R)-1-(2-chloro-6-methylpyridin-4-yl)ethan-1-amine (112 g, 460 mmol), sodium carbonate (146 g, 1.38 mol), and Pd(dppf)Cl₂ (16.8 g, 23.0 mmol) in dioxane (2.24 L) and water (448 mL) was sparged with nitrogen for 5 min and then heated to 80° C. for 16 h. Upon completion, the mixture was cooled to room temperature and filtered. The filter cake was washed with 2-methyltetrahydrofuran (1.0 L), and the filtrate was extracted with 2-methyltetrahydrufuran (7×1500 mL). The combined extracts were then dried over anhydrous magnesium sulfate, filtered, and concentrated. The residue was purified by silica chromatography, eluted with a gradient of methanol in ethyl acetate (0%-50%). The product fractions were concentrated under reduced pressure, and the residue was triturated with a solution of dichloromethane (500 mL) and methyl-tert-butyl ether (500 mL). The precipitate was collected via filtration and to afford (1R)-1-[2-(5-chloro-1-methyl-1H-pyrrolo[2,3-c]pyridin-3-yl)-6-methylpyridin-4-yl]ethan-1-amine (322 g, 46%) as a solid. ¹H NMR (400 MHz, DMSO-d₆) 8.72 (s, 1H), 8.40 (d, J=6.9 Hz, 2H), 7.69-7.59 (m, 1H), 7.08 (s, 1H), 4.07-4.00 (m, 1H), 3.97 (s, 3H), 2.53 (s, 3H), 1.42-1.20 (m, 3H); LCMS m/z for C₁₆H₁₇ClN₄, 300.5) [M+H]⁺.

### PI3K-α Inhibitors

### Example 1

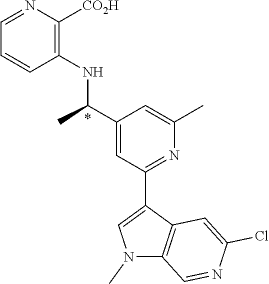 [SMILES: Cc1cc([C@@H](C)Nc2cccnc2C(=O)O)cc(-c2cn(C)c3cnc(Cl)cc23)n1]

### 3-({(1R*)-1-[2-(5-chloro-1-methyl-1H-pyrrolo[2,3-c]pyridin-3-yl)-6-methylpyridin-4-yl]ethyl}amino)pyridine-2-carboxylic Acid

Step 1: To a mixture of (1R*)-1-(2-chloro-6-methylpyridin-4-yl)ethan-1-amine (380 mg, 1.83 mmol) and sodium carbonate (583 mg, 5.50 mmol) in dioxane (10.0 mL) and water (2.0 mL) was added Pd(dppf)Cl₂ (67.1 mg, 0.917 mmol). The mixture was sparged with nitrogen for 1 minute, and then stirred for 10 minutes. 5-Chloro-1-methyl-3-(4,4,5,5-tetramethyl-1,3,2-dioxaborolan-2-yl)-1H-pyrrolo[2,3-c]pyridine (590 mg, 2.02 mmol) was then added to the mixture. The mixture was sparged again for 1 minute, and then heated to 80° C. for 2 hours. The mixture was then cooled to room temperature and partitioned between ethyl acetate (10 mL) and water (5 mL). The aqueous phase was extracted with 1:1 ethyl acetate/THF (2×), and the combined organic extracts were washed with brine, dried over sodium sulfate, and concentrated. Silica gel chromatography eluting with ethyl acetate/hexanes followed by methanol/ethyl acetate afforded (1R*)-1-[2-(5-chloro-1-methyl-1H-pyrrolo[2,3-c]pyridin-3-yl)-6-methylpyridin-4-yl]ethan-1-amine (215 mg, 39%) as a gum. ¹H NMR (400 MHz, (CD₃)₂SO): 8.71 (d, J=0.8 Hz, 1H), 8.42-8.36 (m, 2H), 7.61 (s, 1H), 7.06 (s, 1H), 3.99-3.92 (m, 4H), 2.52 (br s, 3H), 1.28 (d, J=6.6 Hz, 3H); LC-MS m/z for C₁₆H₁₇ClN₄, 301.0 (M+H)⁺.

Step 2: A mixture of (1R*)-1-[2-(5-chloro-1-methyl-1H-pyrrolo[2,3-c]pyridin-3-yl)-6-methylpyridin-4-yl]ethan-1-amine (195 mg, 0.648 mmol), methyl 3-fluoropyridine-2-carboxylate (151 mg, 0.972 mmol), and diisopropylethylamine (251 mg, 1.94 mmol) in anhydrous DMSO (3.0 mL) was heated to 110° C. for 40 hours. The mixture was cooled to room temperature and partitioned between ethyl acetate (10 mL) and water (10 mL). The aqueous phase was extracted with 1:1 ethyl acetate/THF (2×10 mL), and the combined organic extracts were washed with brine, dried over sodium sulfate, and concentrated. Silica gel chromatography eluting with ethyl acetate/hexane followed by methanol/ethyl acetate afforded methyl 3-({(1R*)-1-[2-(5-chloro-1-methyl-1H-pyrrolo[2,3-c]pyridin-3-yl)-6-methylpyridin-4-yl]ethyl}amino)pyridine-2-carboxylate (140 mg, 50%) as a gum. LCMS m/z for C₂₃H₂₂ClN₅O₂, 436.2 (M+H)⁺.

Step 3: To a mixture of methyl 3-({(1R*)-1-[2-(5-chloro-1-methyl-1H-pyrrolo[2,3-c]pyridin-3-yl)-6-methylpyridin-4-yl]ethyl}amino)pyridine-2-carboxylate (140 mg, 0.321 mmol) in THF (5 mL) and water (1.5 mL) was added lithium hydroxide hydrate (40.4 mg, 0.964 mmol). The mixture was heated to 65° C. for 1 hour. The mixture was then cooled to room temperature, neutralized with formic acid, and concentrated. The residue was purified by preparative HPLC on a C18 column, eluting with 0.05% ammonium hydroxide buffer to afford 3-({(1R*)-1-[2-(5-chloro-1-methyl-1H-pyrrolo[2,3-c]pyridin-3-yl)-6-methylpyridin-4-yl]ethyl}amino)pyridine-2-carboxylic acid (60 mg, 44%) as a solid. ¹H NMR (400 MHz, (CD₃)₂SO): 8.71 (s, 1H), 8.65 (br d, J=4.5 Hz, 1H), 8.38 (s, 1H), 8.33 (s, 1H), 7.84 (br d, J=4.1 Hz, 1H), 7.61 (s, 1H), 7.37 (dd, J=4.4, 8.6 Hz, 1H), 7.12-7.03 (m, 2H), 4.73 (quin, J=6.2 Hz, 1H), 3.95 (s, 3H), 2.51 (br s, 3H), 1.57 (d, J=6.6 Hz, 3H); LCMS m/z for C₂₂H₂₀ClN₅O₂, 422.1 (M+H)⁺.

Additional Examples were synthesized by modifications of the methods exemplified above and are shown in Table 1. The Scheme in the table below refers to the scheme number procedure above in which the compound was prepared following a similar procedure with non-critical changes or substitutions to the exemplified procedures that someone who is skilled in the art would be able to realize.

TABLE 1

| Example | | |
|---|---|---|
| # | Structure/Compound Name | Characterizing Data |
| 2 Scheme I | 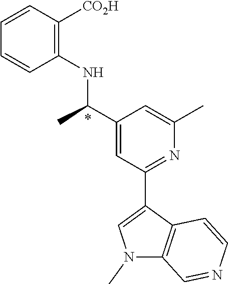 [SMILES: Cc1cc([C@@H](C)Nc2ccccc2C(=O)O)cc(-c2cn(C)c3cnccc23)n1] | ¹H NMR (400 MHz, (CD₃)₂SO) δ 8.94- 8.81 (m, 1H), 8.47-8.29 (m, 1H), 8.27- 8.12 (m, 3H), 7.90-7.78 (m, 1H), 7.62- 7.52 (m, 1H), 7.33-7.17 (m, 1H), 7.06- 6.98 (m, 1H), 6.59-6.46 (m, 2H), 4.76- 4.58 (m, 1H), 4.01-3.92 (m, 3H), 2.49- 2.48 (m, 3H), 1.59-1.49 (m, 3H) LC-MS m/z for C₂₃H₂₂N₄O₂, 387.1 |
| | 2-({(1R*)-1-[2-methyl-6-(1-methyl- | |
| | 1H-pyrrolo[2,3-c]pyridin-3-yl)pyridin- | |
| | 4-yl]ethyl}amino)benzoic acid | |
| 3 Scheme I | 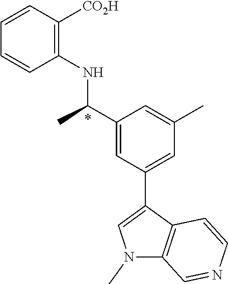 [SMILES: Cc1cc(-c2cn(C)c3cnccc23)cc([C@@H](C)Nc2ccccc2C(=O)O)c1] | ¹H NMR (600 MHz, (CD₃)₂SO) δ 8.85 (s, 1H), 8.17-8.11 (m, 1H), 7.83 (s, 1H), 7.82 (dd, J = 7.9, 1.8 Hz, 1H), 7.60 (d, J = 5.5 Hz, 1H), 7.43 (s, 1H), 7.31 (s, 1H), 7.24-7.20 (m, 1H), 7.07 (s, 1H), 6.63 (d, J = 8.6 Hz, 1H), 6.53 (t, J = 7.5 Hz, 1H), 4.73 (q, J = 6.7 Hz, 1H), 3.93 (s, 3H), 2.35 (s, 3H), 1.54 (d, J = 6.7 Hz, 3H) LC-MS m/z for C₂₄H₂₃N₃O₂, 387.1 |
| | 2-({(1R*)-1-[3-methyl-5-(1-methyl- | |
| | 1H-pyrrolo[2,3-c]pyridin-3- | |
| | yl)phenyl]ethyl}amino)benzoic acid | |
| 4 Scheme I | 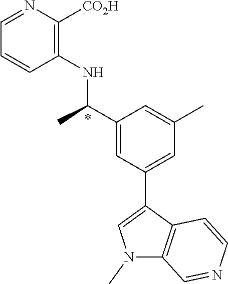 [SMILES: Cc1cc(-c2cn(C)c3cnccc23)cc([C@@H](C)Nc2cccnc2C(=O)O)c1] | ¹H NMR (400 MHz, (CD₃)₂SO) δ 8.87 (s, 1H), 8.81-8.66 (m, 1H), 8.22-8.15 (m, 2H), 7.92 (s, 1H), 7.82 (d, J = 4.0 Hz, 1H), 7.66 (d, J = 5.6 Hz, 1H), 7.45 (s, 1H), 7.41- 7.32 (m, 2H), 7.17 (d, J = 8.7 Hz, 1H), 7.07 (s, 1H), 4.78 (quin, J = 6.5 Hz, 1H), 3.94 (s, 3H), 2.34 (s, 3H), 1.56 (d, J = 6.6 Hz, 3H) LCMS m/z (ESI+) for C₂₃H₂₂N₄O₂, 387.2 |
| | 3-({(1R*)-1-[3-methyl-5-(1-methyl- | |
| | 1H-pyrrolo[2,3-c]pyridin-3- | |
| | yl)phenyl]ethyl}amino)pyridine-2- | |
| | carboxylic acid | |
| 5 Scheme I | 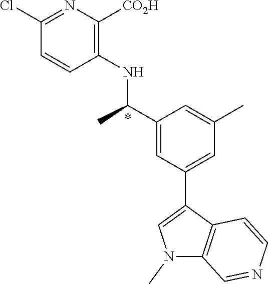 [SMILES: Cc1cc(-c2cn(C)c3cnccc23)cc([C@@H](C)Nc2ccc(Cl)nc2C(=O)O)c1] | ¹H NMR (400 MHz, (CD₃)₂SO) δ 8.89 (s, 1H), 8.40 (br d, J = 6.2 Hz, 1H), 8.18 (d, J = 5.5 Hz, 1H), 7.93 (s, 1H), 7.68 (dd, J = 0.8, 5.7 Hz, 1H), 7.43 (s, 1H), 7.38-7.33 (m, 2H), 7.16 (d, J = 9.0 Hz, 1H), 7.05 (s, 1H), 4.78 (br t, J = 6.3 Hz, 1H), 3.94 (s, 3H), 2.34 (s, 3H), 1.55 (d, J = 6.7 Hz, 3H) LCMS m/z (ESI+) for C₂₃H₂₁ClN₄O₂, 421.0 (M + H)⁺ |
| | 6-chloro-3-({(1R*)-1-[3-methyl-5-(1- | |
| | methyl-1H-pyrrolo[2,3-c]pyridin-3- | |
| | yl)phenyl]ethyl}amino)pyridine-2- | |
| | carboxylic acid | |
| 6 Scheme II | 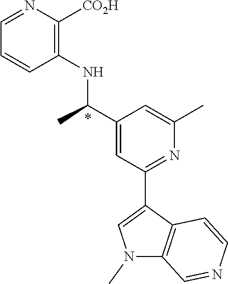 [SMILES: Cc1cc([C@@H](C)Nc2cccnc2C(=O)O)cc(-c2cn(C)c3cnccc23)n1] | ¹H NMR (400 MHz, DMSO-d₆) δ 8.84 (s, 1H), 8.24-8.17 (m, 3H), 7.77 (d, J = 4.5 Hz, 1H), 7.54 (s, 1H), 7.45 (dd, J = 5.0, 8.6 Hz, 1H), 7.29 (br d, J = 8.7 Hz, 1H), 7.05 (s, 1H), 4.80-4.70 (m, 1H), 3.92 (s, 3H), 2.47 (s, 3H), 1.54 (br d, J = 6.6 Hz, 3H) LCMS m/z (ESI+) for C₂₂H₂₁N₅O₂, 388.2 [M + H]⁺ |
| | 3-({(1R*)-1-[2-methyl-6-(1-methyl- | |
| | 1H-pyrrolo[2,3-c]pyridin-3-yl)pyridin- | |
| | 4-yl]ethyl}amino)pyridine-2- | |
| | carboxylic acid | |

### Example 7

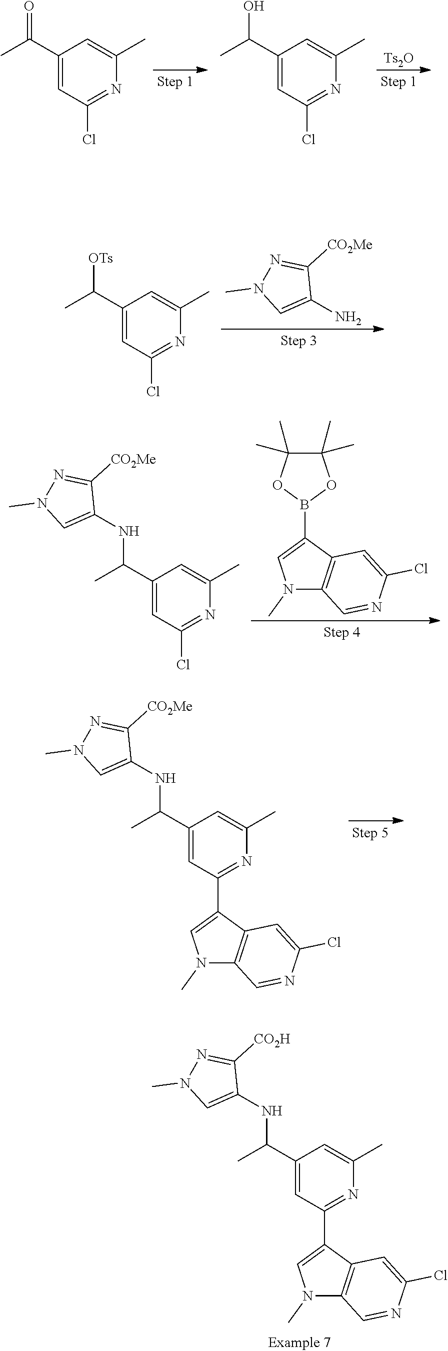

### 4-({1-[2-(5-chloro-1-methyl-1H-pyrrolo[2,3-c]pyridin-3-yl)-6-methylpyridin-4-yl]ethyl}amino)-1-methyl-1H-pyrazole-3-carboxylic acid

Step 1: To a solution of 1-(2-chloro-6-methylpyridin-4-yl)ethan-1-one (1.10 g, 6.17 mmol) in methanol (15.4 mL) at 0° C. was added portionwise sodium borohydride (350 mg, 9.26 mmol). After 2 min, the mixture was warmed to room temperature and stirred for 10 minutes. Upon completion, the mixture was cooled to 0° C., quenched with water, and adjusted to PH˜9 with 1M HCl. The mixture was then extracted with an equal volume of dichloromethane (3×) and with an equal volume of ethyl acetate (1×). The combined extracts were then dried over anhydrous sodium sulfate, filtered, and concentrated to afford 1-(2-chloro-6-methylpyridin-4-yl)ethan-1-ol as an oil. The material was used in the next step without further purification. ¹H NMR (400 MHz, DMSO-d₆) δ 7.22 (d, J=2.5 Hz, 2H), 5.45 (d, J=4.6 Hz, 1H), 4.75-4.65 (m, 1H), 2.43 (s, 3H), 1.31 (d, J=6.6 Hz, 3H); LCMS m/z for C₈H₁₀ClNO, 172.0 (M+H)⁺.

Step 2: To a solution of 1-(2-chloro-6-methylpyridin-4-yl)ethan-1-ol and N,N-diisopropylethylamine (203 mg, 0.270 mL) in dichloromethane (3.00 mL) at 0° C. was added p-toluenesulfonic anhydride (513 mg, 1.57 mmol), and the resultant mixture was allowed to warm to room temperature. To the mixture was then added triethylamine (160 mg, 0.220 mL) and additional p-toluenesulfonic anhydride (250 mg, 0.76 mmol), followed by heating to 35° C. for 1 h. The mixture was then cooled to rt, quenched with water, and stirred for 10 minutes. The layers were divided, and the aqueous portion was extracted twice with dichloromethane. The combined organic extracts were dried over anhydrous sodium sulfate, filtered, and concentrated. The residue was purified by silica gel chromatography, eluted with a gradient of ethyl acetate in heptane (0%-100% over 25 min) to afford 1-(2-chloro-6-methylpyridin-4-yl)ethyl 4-methylbenzene-1-sulfonate as an oil. ¹H NMR (400 MHz, DMSO-d₆) δ 7.74-7.68 (m, 2H), 7.42-7.37 (m, 2H), 7.12 (s, 2H), 5.59 (q, J=6.7 Hz, 1H), 2.39 (s, 3H), 2.38 (s, 3H), 1.46 (d, J=6.6 Hz, 3H); LCMS m/z for C₁₅H₁₆ClNO₃S, 326.1 (M+H)⁺.

Step 3: To a solution of 1-(2-chloro-6-methylpyridin-4-yl)ethyl 4-methylbenzene-1-sulfonate (50 mg, 0.15 mmol) and methyl 4-amino-1-methyl-1H-pyrazole-3-carboxylate (29 mg, 0.18 mmol) in acetonitrile (0.30 mL) was added potassium carbonate (25.0 mg, 0.18 mmol), and the resultant mixture was heated to 80° C. for ˜3 h. The mixture was then cooled to room temperature and found to be incomplete. Additional methyl 4-amino-1-methyl-1H-pyrazole-3-carboxylate (10.0 mg, 0.06 mmol) and potassium carbonate (8.0 mg, 0.06 mmol) were added, and heating was resumed for an additional 3.5 h. The mixture was then cooled to room temperature and partitioned between water, brine, and ethyl acetate. The organic layer was dried over anhydrous sodium sulfate, filtered, and concentrated. The residue was purified by silica chromatography, eluted with a gradient of ethyl acetate in heptane (10%-100% over 15 min) to afford methyl 4-{[1-(2-chloro-6-methylpyridin-4-yl)ethyl]amino}-1-methyl-1H-pyrazole-3-carboxylate as a solid. ¹H NMR (400 MHz, DMSO-d₆) δ 7.29-7.23 (m, 2H), 6.94 (s, 1H), 5.32 (d, J=7.1 Hz, 1H), 4.22 (p, J=6.9 Hz, 1H), 3.79 (s, 3H), 3.70 (s, 3H), 2.42 (s, 3H), 1.43 (d, J=6.8 Hz, 3H); LCMS m/z for C₁₄H₁₇ClN₄O₂, 310.1 (M+H)⁺.

Step 4: A mixture of methyl 4-{[1-(2-chloro-6-methylpyridin-4-yl)ethyl]amino}-1-methyl-1H-pyrazole-3-carboxylate (70.0 mg, 0.230 mmol), 5-chloro-1-methyl-3-(4,4,5,5-tetramethyl-1,3,2-dioxaborolan-2-yl)-1H-pyrrolo[2,3-c]pyridine (82.0 mg, 0.280 mmol), sodium bicarbonate (60.0 mg, 0.710 mmol), and tetrakis(triphenylphosphine) palladium (0) (53.0 mg, 46.0 μmol) in a solution of tert-amyl alcohol (0.90 mL), 1,4-dioxane (0.40 mL), and water (0.18 mL) at room temperature was sparged with nitrogen for 5 minutes, and the resultant mixture was then heated to 95° C. for several hours. Upon completion, the mixture was diluted with water and extracted twice with ethyl acetate. The combined extracts were then concentrated, and the resultant residue was purified by silica gel chromatography, eluted with a gradient of methanol/acetonitrile (1:3) in dichloromethane (0%-80% over 20 min) to afford methyl 4-({1-[2-(5-chloro-1-methyl-1H-pyrrolo[2,3-c]pyridin-3-yl)-6-methylpyridin-4-yl]ethyl}amino)-1-methyl-1H-pyrazole-3-carboxylate as a solid. ¹H NMR (400 MHz, DMSO-d₆) δ 8.71 (d, J=0.9 Hz, 1H), 8.35 (s, 1H), 8.33 (d, J=0.9 Hz, 1H), 7.60-7.58 (m, 1H), 7.05 (d, J=1.3 Hz, 1H), 6.96 (s, 1H), 5.34 (d, J=6.9 Hz, 1H), 4.27-4.19 (m, 1H), 3.96 (s, 3H), 3.81 (s, 3H), 3.69 (s, 3H), 2.52 (s, 3H), 1.49 (d, J=6.8 Hz, 3H); LCMS m/z for C₂₂H₂₃ClN₆O₂, 439.3 (M+H)⁺.

Step 5: To a solution of methyl 4-({1-[2-(5-chloro-1-methyl-1H-pyrrolo[2,3-c]pyridin-3-yl)-6-methylpyridin-4-yl]ethyl}amino)-1-methyl-1H-pyrazole-3-carboxylate (90.0 mg, 0.210 mmol) in tetrahydrofuran (1.5 mL) at room temperature was added a solution of lithium hydroxide (39.0 mg, 1.60 mmol) in water (0.50 mL), and the resultant mixture was then heated to 60° C. for 30 minutes. Additional lithium hydroxide (14.0 mg, 0.58 mmol) was then added, and the mixture was heated for another 50 minutes. Upon completion, the mixture was cooled to room temperature and diluted with a 4.0M solution of hydrochloric acid (58 mg, 0.40 mL). The mixture was stirred until a clear solution resulted and was then concentrated under a stream of nitrogen. The residue was purified be reversed-phase HPLC (column: Phenomenex Gemini 5 μm NX-C18 Dimensions: 21.2 mm×150 mm; mobile phase A: water (10 mM NH₄OAc); mobile phase B: acetonitrile; gradient: 10%-50% B over 8.0 min; flow rate: 25 mL/min), to afford 4-({1-[2-(5-chloro-1-methyl-1H-pyrrolo[2,3-c]pyridin-3-yl)-6-methylpyridin-4-yl]ethyl}amino)-1-methyl-1H-pyrazole-3-carboxylic acid (21.4 mg, >99%) as a powder. ¹H NMR (600 MHz, DMSO-d₆) δ 8.65 (d, J=2.0 Hz, 1H), 8.31-8.28 (m, 1H), 8.23-8.21 (m, 1H), 7.53 (d, J=2.4 Hz, 1H), 7.02 (s, 1H), 6.89 (s, 1H), 4.22 (q, J=6.8 Hz, 1H), 3.94 (d, J=1.7 Hz, 3H), 3.66 (s, 3H), 2.51 (d, J=1.8 Hz, 4H), 1.49-1.44 (m, 3H); LCMS m/z for C₂₁H₂₁ClN₆O₂, 425.3 (M+H)⁺.

### Example 8

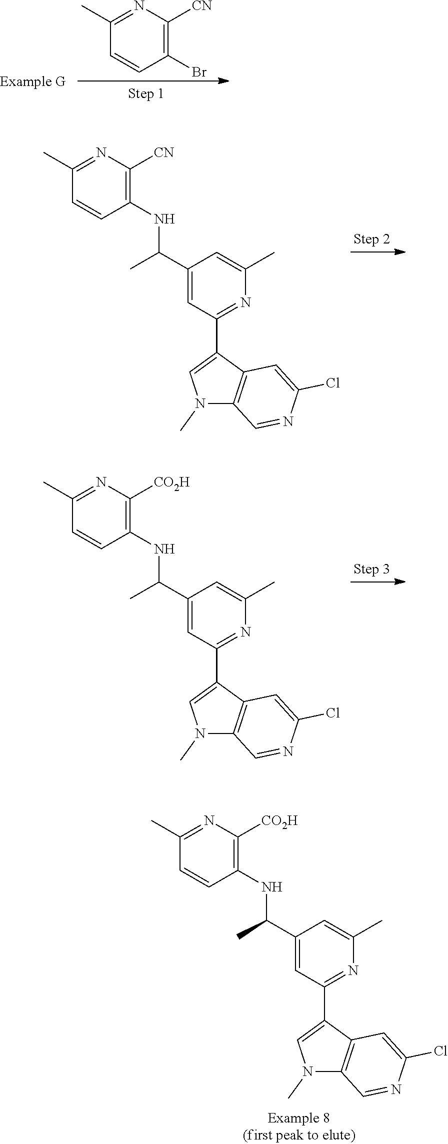

### 3-({(1R)-1-[2-(5-chloro-1-methyl-1H-pyrrolo[2,3-c]pyridin-3-yl)-6-methylpyridin-4-yl]ethyl}amino)-6-methylpyridine-2-carboxylic Acid

Step 1: To a mixture of (1R)-1-[2-(5-chloro-1-methyl-1H-pyrrolo[2,3-c]pyridin-3-yl)-6-methylpyridin-4-yl]ethan-1-amine (180 mg, 0.598 mmol), 3-bromo-6-methylpyridine-2-carbonitrile (124 mg, 0.628 mmol), and cesium carbonate (585 mg, 1.80 mmol) in 1,4-dioxane (8.0 mL) at room temperature was added XantPhosPd G3 (30.9 mg, 0.0299 mmol), and the resultant mixture was then purged with nitrogen and heated to 100° C. for 16 h. Upon completion, the mixture was filtered through a pad of diatomaceous earth, and the filter cake was washed with tetrahydrofuran (3×10 mL). The filtrate was then concentrated under vacuum, and the residue was purified by silica gel chromatography eluted with a gradient of ethyl acetate in n-hexane (0%-75%) to afford 3-({1-[2-(5-chloro-1-methyl-1H-pyrrolo[2,3-c]pyridin-3-yl)-6-methylpyridin-4-yl]ethyl}amino)-6-methylpyridine-2-carbonitrile as a solid (225 mg, 90% yield). LCMS m/z for C₂₃H₂₁ClN₆, 417.1 [M+H]⁺.

Step 2: To a mixture of 3-({1-[2-(5-chloro-1-methyl-1H-pyrrolo[2,3-c]pyridin-3-yl)-6-methylpyridin-4-yl]ethyl}amino)-6-methylpyridine-2-carbonitrile (225 mg, 0.540 mmol) in ethanol (2.5 mL) at room temperature was added a solution of sodium hydroxide (648 mg, 16.2 mmol) in water (2.5 mL), and the resultant mixture was then heated to 100° C. for 16 h. Upon completion, the mixture was adjusted to pH˜8, and the resultant precipitate was collected via filtration and rinsed with water (˜10 mL). The solid material was further purified by preparative HPLC then dissolved in dimethyl sulfoxide, and subjected to preparative high-performance liquid chromatography (column: 52-Welch Xtimate C18 column (150×30 mm, 5 μm); mobile phase A: water (0.225% formic acid); mobile phase B: acetonitrile; gradient: 0%-35% acetonitrile over 10 min; flow rate: 30 mL/min, detection: of 220 and 254 nm) to afford 3-({1-[2-(5-chloro-1-methyl-1H-pyrrolo[2,3-c]pyridin-3-yl)-6-methylpyridin-4-yl]ethyl}amino)-6-methylpyridine-2-carboxylic acid as a solid (58.1 mg, 25%). ¹H NMR (400 MHz, DMSO-d₆): 8.71 (d, J=0.6 Hz, 1H), 8.41 (br d, J=5.4 Hz, 1H), 8.37 (s, 1H), 8.32 (d, J=0.7 Hz, 1H), 7.59 (s, 1H), 7.24 (d, J=8.8 Hz, 1H), 7.08-6.98 (m, 2H), 4.76-4.66 (m, 1H), 3.95 (s, 3H), 2.50 (s, 3H), 2.35 (s, 3H), 1.55 (d, J=6.7 Hz, 3H); LCMS m/z for C₂₃H₂₁ClN₅O₂, 436.2 [M+H]⁺.

Step 3: Chiral separation of 3-({1-[2-(5-chloro-1-methyl-1H-pyrrolo[2,3-c]pyridin-3-yl)-6-methylpyridin-4-yl]ethyl}amino)-6-methylpyridine-2-carboxylic acid (114 mg) was accomplished by chiral HPLC (column: Chiralpak IK, 21 mm×250 mm and 5 μm particle size; mobile phase A: methanol (0.1% acetic acid)/acetonitrile (98:2); flow rate 15 mL/min) to afford 3-({(1R)-1-[2-(5-chloro-1-methyl-1H-pyrrolo[2,3-c]pyridin-3-yl)-6-methylpyridin-4-yl]ethyl}amino)-6-methylpyridine-2-carboxylic acid as the first peak: ¹H NMR (600 MHz, DMSO) δ 8.70 (d, J=0.9 Hz, 1H), 8.41 (d, J=6.5 Hz, 1H), 8.34 (s, 1H), 8.29 (d, J=1.0 Hz, 1H), 7.57 (d, J=1.4 Hz, 1H), 7.25 (d, J=8.8 Hz, 1H), 7.06 (d, J=8.8 Hz, 1H), 7.03 (d, J=1.4 Hz, 1H), 4.71 (td, J=7.7, 5.5 Hz, 1H), 3.95 (s, 3H), 2.35 (s, 3H), 1.55 (d, J=6.7 Hz, 3H), and second peak: ¹H NMR (600 MHz, DMSO) δ 8.70 (d, J=1.0 Hz, 1H), 8.42 (d, J=6.5 Hz, 1H), 8.33 (s, 1H), 8.29 (d, J=1.1 Hz, 1H), 7.57 (s, 1H), 7.24 (d, J=8.8 Hz, 1H), 7.06 (d, J=8.8 Hz, 1H), 7.03 (d, J=1.4 Hz, 1H), 4.71 (td, J=6.8, 4.6 Hz, 1H), 3.94 (s, 3H), 2.35 (s, 3H), 1.55 (d, J=6.7 Hz, 3H).

Additional Examples were synthesized by modifications of the methods exemplified above and are shown in Table 2. The Scheme in the table below refers to the scheme number procedure above in which the compound was prepared following a similar procedure with non-critical changes or substitutions to the exemplified procedures that someone who is skilled in the art would be able to realize.

TABLE 2

| Example | | |
|---|---|---|
| # | Structure/Compound Name | Characterizing Data |
| 9 Scheme I | 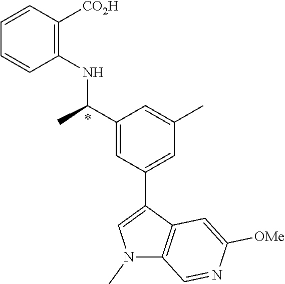 [SMILES: COc1cc2c(-c3cc(C)cc([C@@H](C)Nc4ccccc4C(=O)O)c3)cn(C)c2cn1] | ¹H NMR (400 MHz, DMSO-d₆): 12.68 (br s, 1H), 8.47 (s, 1H), 8.34 (br d, J = 5.3 Hz, 1H), 7.88 (s, 1H), 7.79 (dd, J = 1.5, 7.9 Hz, 1H), 7.41 (s, 1H), 7.29 (s, 1H), 7.26- 7.19 (m, 1H), 7.03 (s, 2H), 6.58 (d, J = 8.4 Hz, 1H), 6.51 (t, J = 7.2 Hz, 1H), 4.75- 4.64 (m, 1H), 3.85 (d, J = 8.2 Hz, 6H), 2.33 (s, 3H), 1.53 (d, J = 6.7 Hz, 3H) LCMS m/z (ESI) for C₂₅H₂₅N₃O₃, 416.2 [M + H]⁺ |
| | 2-({(1R*)-1-[3-(5-methoxy-1-methyl- | |
| | 1H-pyrrolo[2,3-c]pyridin-3-yl)-5- | |
| | methylphenyl]ethyl}amino)benzoic | |
| | acid | |
| 10 Scheme II | 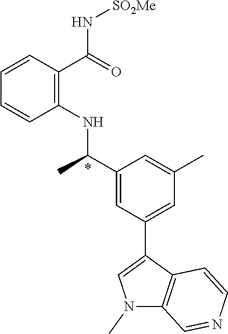 [SMILES: Cc1cc(-c2cn(C)c3cnccc23)cc([C@@H](C)Nc2ccccc2C(=O)NS(C)(=O)=O)c1] | ¹H NMR (400 MHz, DMSO-d₆) δ 9.09 (br s, 1H), 8.27-8.20 (m, 2H), 7.90 (br d, J = 5.7 Hz, 1H), 7.83 (br d, J = 8.4 Hz, 1H), 7.50 (s, 1H), 7.35 (s, 1H), 7.18-7.11 (m, 2H), 6.55 (d, J = 8.6 Hz, 1H), 6.49 (t, J = 7.4 Hz, 1H), 4.71 (br d, J = 7.0 Hz, 1H), 4.02-3.99 (m, 3H), 3.17 (s, 3H), 2.36 (s, 3H), 1.53 (br d, J = 6.6 Hz, 3H) LCMS m/z (ESI) for C₂₅H₂₆N₄O₃S, 463.2 [M + H]⁺ |
| | N-(methanesulfonyl)-2-({(1R)-1-[3- | |
| | methyl-5-(1-methyl-1H-pyrrolo[2,3- | |
| | c]pyridin-3- | |
| | yl)phenyl]ethyl}amino)benzamide | |
| 11 Scheme I | 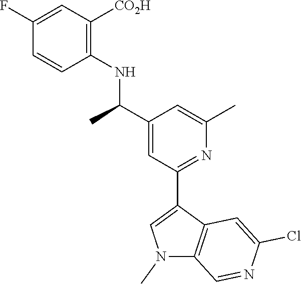 [SMILES: Cc1cc([C@@H](C)Nc2ccc(F)cc2C(=O)O)cc(-c2cn(C)c3cnc(Cl)cc23)n1] | ¹H NMR (400 MHz, DMSO-d₆) δ 13.58- 12.76 (m, 1H), 8.71 (d, J = 0.6 Hz, 1H), 8.36 (s, 1H), 8.28 (d, J = 0.6 Hz, 2H), 7.58 (s, 1H), 7.53 (dd, J = 3.3, 9.8 Hz, 1H), 7.15 (dt, J = 3.3, 8.6 Hz, 1H), 7.03 (s, 1H), 6.48 (dd, J = 4.5, 9.4 Hz, 1H), 4.65 (br d, J = 6.6 Hz, 1H), 3.95 (s, 3H), 2.52-2.51 (m, 3H), 1.53 (d, J = 6.8 Hz, 3H) LCMS m/z (ESI) for C₂₃H₂₀ClN₄O₂, 439.2 [M + H]⁺ |
| | 2-({(1R)-1-[2-(5-chloro-1-methyl-1H- | |
| | pyrrolo[2,3-c]pyridin-3-yl)-6- | |
| | methylpyridin-4-yl]ethyl}amino)-5- | |
| | fluorobenzoic acid | |
| 12 Scheme I | 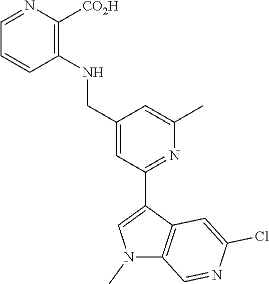 [SMILES: Cc1cc(CNc2cccnc2C(=O)O)cc(-c2cn(C)c3cnc(Cl)cc23)n1] | ¹H NMR (400 MHz, DMSO-d₆,) δ 8.72 (s, 1H), 8.65 (br s, 1H), 8.34 (d, J = 12.2 Hz, 2H), 7.89 (d, J = 4.0 Hz, 1H), 7.57 (s, 1H), 7.42 (dd, J = 4.3, 8.6 Hz, 1H), 7.21 (d, J = 8.4 Hz, 1H), 7.02 (s, 1H), 4.59 (br d, J = 5.3 Hz, 2H), 3.96 (s, 3H), 2.54-2.52 (m, 3H) |
| | 3-({[2-(5-chloro-1-methyl-1H- | |
| | pyrrolo[2,3-c]pyridin-3-yl)-6- | |
| | methylpyridin-4- | |
| | yl]methyl}amino)pyridine-2- | |
| | carboxylic acid | |
| 13 Scheme I | 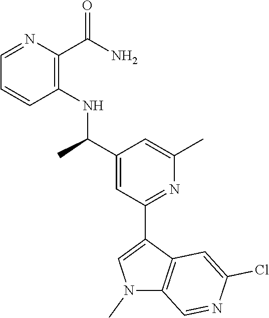 [SMILES: Cc1cc([C@@H](C)Nc2cccnc2C(N)=O)cc(-c2cn(C)c3cnc(Cl)cc23)n1] | ¹H NMR (400 MHz, DMSO-d₆) δ 9.02 (d, J = 6.4 Hz, 1H), 8.71 (s, 1H), 8.37 (s, 1H), 8.32 (s, 1H), 8.13 (br d, J = 2.6 Hz, 1H), 7.78 (dd, J = 1.1, 4.3 Hz, 1H), 7.60 (s, 1H), 7.53 (br d, J = 2.5 Hz, 1H), 7.22 (dd, J = 4.3, 8.5 Hz, 1H), 7.04 (s, 1H), 6.93 (d, J = 7.8 Hz, 1H), 4.66 (quin, J = 6.7 Hz, 1H), 3.95 (s, 3H), 2.50 (s, 3H), 1.54 (d, J = 6.8 Hz, 3H) LCMS m/z (ESI) for C₂₂H₂₁ClN₆O, 421.2 [M + H]⁺ |
| | 3-({(1R)-1-[2-(5-chloro-1-methyl-1H- | |
| | pyrrolo[2,3-c]pyridin-3-yl)-6- | |
| | methylpyridin-4- | |
| | yl]ethyl}amino)pyridine-2- | |
| | carboxamide | |
| 14 Scheme III | 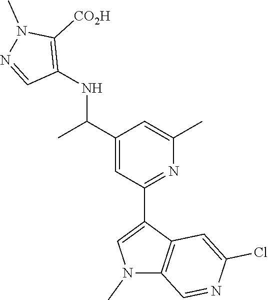 [SMILES: Cc1cc(C(C)Nc2cnn(C)c2C(=O)O)cc(-c2cn(C)c3cnc(Cl)cc23)n1] | LCMS m/z (ESI) for C₂₁H₂₁ClN₆O₂, 425.08 [M + H]⁺ |
| | 4-({1-[2-(5-chloro-1-methyl-1H- | |
| | pyrrolo[2,3-c]pyridin-3-yl)-6- | |
| | methylpyridin-4-yl]ethyl}amino)-1- | |
| | methyl-1H-pyrazole-5-carboxylic acid | |
| 15 Scheme I | 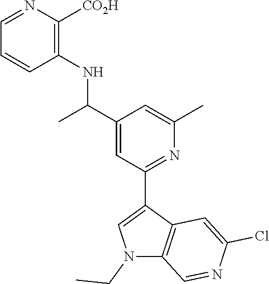 [SMILES: CCn1cc(-c2cc(C(C)Nc3cccnc3C(=O)O)cc(C)n2)c2cc(Cl)ncc21] | ¹H NMR (400 MHz, DMSO-d₆) δ 8.77 (s, 2H), 8.46 (s, 1H), 8.35 (s, 1H), 7.83 (d, J = 3.8 Hz, 1H), 7.65 (s, 1H), 7.33 (dd, J = 4.4, 8.6 Hz, 1H), 7.08-6.99 (m, 2H), 4.70 (br d, J = 5.9 Hz, 1H), 4.37 (q, J = 7.2 Hz, 2H), 2.50 (s, 3H), 1.57 (d, J = 6.8 Hz, 3H), 1.45 (t, J = 7.3 Hz, 3H) LCMS m/z (ESI) for C₂₃H₂₂ClN₅O₂, 436.2 [M + H]⁺ |
| | 3-({1-[2-(5-chloro-1-ethyl-1H- | |
| | pyrrolo[2,3-c]pyridin-3-yl)-6- | |
| | methylpyridin-4- | |
| | yl]ethyl}amino)pyridine-2-carboxylic | |
| | acid | |
| 16 Scheme I | 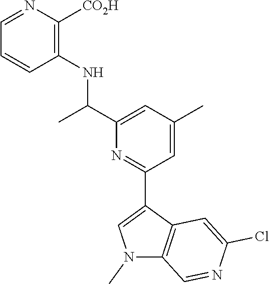 [SMILES: Cc1cc(-c2cn(C)c3cnc(Cl)cc23)nc(C(C)Nc2cccnc2C(=O)O)c1] | LCMS m/z for C₂₂H₂₀ClN₂O₃, 422.2 [M + H]⁺ |
| | 3-({1-[6-(5-chloro-1-methyl-1H- | |
| | pyrrolo[2,3-c]pyridin-3-yl)-4- | |
| | methylpyridin-2- | |
| | yl]ethyl}amino)pyridine-2-carboxylic | |
| | acid | |
| 17 Scheme III | 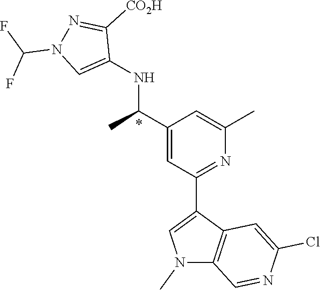 [SMILES: Cc1cc([C@@H](C)Nc2cn(C(F)F)nc2C(=O)O)cc(-c2cn(C)c3cnc(Cl)cc23)n1] | ¹H NMR (400 MHz, DMSO-d₆) δ 8.72 (s, 1H), 8.37 (d, J = 5.1 Hz, 2H), 7.83-7.47 (m, 2H), 7.37 (s, 1H), 7.09 (s, 1H), 4.32 (br d, J = 6.6 Hz, 1H), 3.96 (s, 3H), 2.52 (br s, 3H), 1.51 (br d, J = 6.8 Hz, 3H) LCMS m/z (ESI) for C₂₁H₁₉ClF2N₆O₂, 461.3 [M + H]⁺ [α] = −100° (c 0.1, MeOH) |
| | 4-({(1R*)-1-[2-(5-chloro-1-methyl- | |
| | 1H-pyrrolo[2,3-c]pyridin-3-yl)-6- | |
| | methylpyridin-4-yl]ethyl}amino)-1- | |
| | (difluoromethyl)-1H-pyrazole-3- | |
| | carboxylic acid | |
| 18 Scheme III | 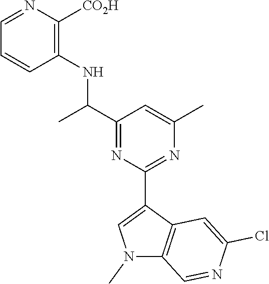 [SMILES: Cc1cc(C(C)Nc2cccnc2C(=O)O)nc(-c2cn(C)c3cnc(Cl)cc23)n1] | LCMS m/z for C₂₁H₁₉ClN₆O₂, 423.1 [M + H]⁺ |
| | 3-({1-[2-(5-chloro-1-methyl-1H- | |
| | pyrrolo[2,3-c]pyridin-3-yl)-6- | |
| | methylpyrimidin-4- | |
| | yl]ethyl}amino)pyridine-2-carboxylic | |
| | acid | |
| 19 Scheme II | 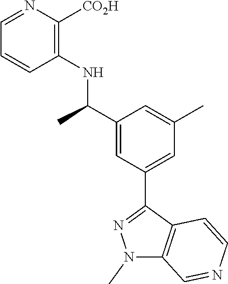 [SMILES: Cc1cc(-c2nn(C)c3cnccc23)cc([C@@H](C)Nc2cccnc2C(=O)O)c1] | ¹H NMR (400 MHz, DMSO-d₆) δ 9.21 (s, 1H), 8.69 (br d, J = 6.6 Hz, 1H), 8.30 (d, J = 5.8 Hz, 1H), 8.13 (s, 1H), 7.96 (dd, J = 0.8, 5.7 Hz, 1H), 7.82 (d, J = 3.6 Hz, 1H), 7.76 (s, 1H), 7.68 (s, 1H), 7.38 (dd, J = 4.4, 8.7 Hz, 1H), 7.24 (s, 1H), 7.19 (d, J = 8.4 Hz, 1H), 4.85 (quin, J = 6.5 Hz, 1H), 4.22 (s, 3H), 2.39 (s, 3H), 1.57 (d, J = 6.6 Hz, 3H) LCMS m/z (ESI) for C₂₂H₂₁N₅O₂, 388.2 [M + H]⁺ |
| | 3-({(1R)-1-[3-methyl-5-(1-methyl- | |
| | 1H-pyrazolo[3,4-c]pyridin-3- | |
| | yl)phenyl]ethyl}amino)pyridine-2- | |
| | carboxylic acid | |
| 20 Scheme I | 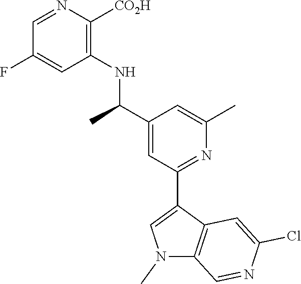 | ¹H NMR (400 MHz, DMSO-d₆) δ 8.72 (s, 1H), 8.53 (br d, J = 5.1 Hz, 1H), 8.36 (d, J = 17.9 Hz, 2H), 7.84 (s, 1H), 7.63 (s, 1H), 7.07 (s, 1H), 6.90 (br d, J = 11.6 Hz, 1H), 4.82-4.67 (m, 1H), 3.96 (s, 3H), 2.52 (br s, 3H), 1.62-1.49 (m, 3H) LCMS m/z (ESI) for C₂₂H₁₉N₅O₂, 400.2 [M + H]⁺ |
| | 3-({(1R)-1-[2-(5-chloro-1-methyl-1H- | |
| | pyrrolo[2,3-c]pyridin-3-yl)-6- | |
| | methylpyridin-4-yl]ethyl}amino)-5- | |
| | fluoropyridine-2-carboxylic acid | |
| 21 Scheme I | 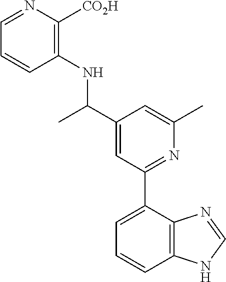 [SMILES: Cc1cc(C(C)Nc2cccnc2C(=O)O)cc(-c2cccc3[nH]cnc23)n1] | LCMS m/z for C₂₁H₁₉N₅O₂, 372.2 [M − H]⁻ |
| | 3-({1-[2-(1H-1,3-benzimidazol-4-yl)- | |
| | 6-methylpyridin-4- | |
| | yl]ethyl}amino)pyridine-2-carboxylic | |
| | acid | |
| 22 Scheme II | 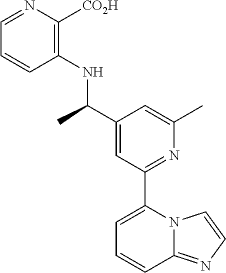 [SMILES: Cc1cc([C@@H](C)Nc2cccnc2C(=O)O)cc(-c2cccc3nccn23)n1] | LCMS m/z for C₂₁H₁₉N₅O₂, 374.2 [M + H]⁺ |
| | 3-({(1R)-1-[2-(imidazo[1,2-a]pyridin- | |
| | 5-yl)-6-methylpyridin-4- | |
| | yl]ethyl}amino)pyridine-2-carboxylic | |
| | acid | |
| 23 Scheme III | 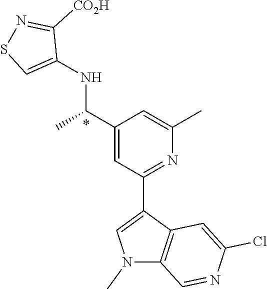 [SMILES: Cc1cc([C@H](C)Nc2csnc2C(=O)O)cc(-c2cn(C)c3cnc(Cl)cc23)n1] | ¹H NMR (400 MHz, DMSO-d₆) δ 8.80- 8.68 (m, 1H), 8.38 (s, 1H), 8.35 (d, J = 0.6 Hz, 1H), 7.70-7.54 (m, 1H), 7.39 (s, 1H), 7.09-7.00 (m, 1H), 4.55-4.30 (m, 1H), 3.95 (s, 3H), 2.51 (br s, 3H), 1.53 (d, J = 6.7 Hz, 3H) LCMS m/z (ESI) for C₂₀H₁₈ClN₅O₂S, 428.1 [M + H]⁺ [α] = −112° (c 0.04, MeOH) |
| | 4-({(1S)-1-[2-(5-chloro-1-methyl-1H- | |
| | pyrrolo[2,3-c]pyridin-3-yl)-6- | |
| | methylpyridin-4-yl]ethyl}amino)-1,2- | |
| | thiazole-3-carboxylic acid | |
| 24 Scheme I | 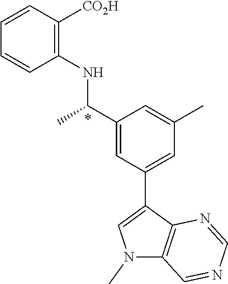 [SMILES: Cc1cc(-c2cn(C)c3cncnc23)cc([C@H](C)Nc2ccccc2C(=O)O)c1] | ¹H NMR (400 MHz, DMSO-d₆) δ 13.06- 12.29 (m, 1H), 9.09 (s, 1H), 8.95 (s, 1H), 8.36 (s, 2H), 7.96 (s, 1H), 7.92 (s, 1H), 7.80 (dd, J = 1.5, 7.9 Hz, 1H), 7.22 (t, J = 7.2 Hz, 1H), 7.04 (s, 1H), 6.58 (d, J = 8.4 Hz, 1H), 6.51 (t, J = 7.5 Hz, 1H), 4.65 (br dd, J = 1.8, 3.3 Hz, 1H), 3.97 (s, 3H), 2.35 (s, 3H), 1.55 (d, J = 6.6 Hz, 3H). LCMS m/z (ESI) for C₂₃H₂₂N₄O₂, 387.1 [M + H]⁺ |
| | 2-({(1S*)-1-[3-methyl-5-(5-methyl- | |
| | 5H-pyrrolo[3,2-d]pyrimidin-7- | |
| | yl)phenyl]ethyl}amino)benzoic acid | |
| 25 Scheme I | 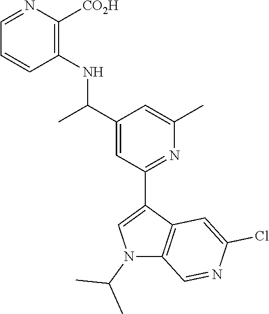 [SMILES: Cc1cc(C(C)Nc2cccnc2C(=O)O)cc(-c2cn(C(C)C)c3cnc(Cl)cc23)n1] | LCMS m/z (ESI) for C₂₄H₂₄ClN₅O₂, 450.3 [M + H]⁺ |
| | 3-[(1-{2-[5-chloro-1-(propan-2-yl)- | |
| | 1H-pyrrolo[2,3-c]pyridin-3-yl]-6- | |
| | methylpyridin-4- | |
| | yl}ethyl)amino]pyridine-2-carboxylic | |
| | acid | |
| 26 Scheme I | 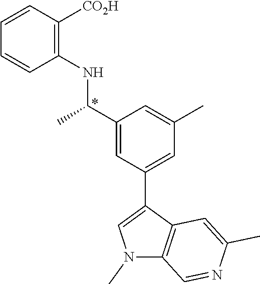 [SMILES: Cc1cc(-c2cn(C)c3cnc(C)cc23)cc([C@H](C)Nc2ccccc2C(=O)O)c1] | ¹H NMR (400 MHz, DMSO-d₆): 8.72 (s, 1H), 8.40 (br s, 1H), 7.87-7.79 (m, 2H), 7.41 (s, 1H), 7.37 (s, 1H), 7.30 (s, 1H), 7.23 (t, J = 7.8 Hz, 1H), 7.06 (s, 1H), 6.61- 6.48 (m, 2H), 4.72 (br d, J = 6.4 Hz, 1H), 3.89 (s, 3H), 2.49 (br s, 3H), 2.35 (s, 3H), 1.53 (d, J = 6.5 Hz, 3H) LCMS m/z (ESI) for C₂₅H₂₅N₃O₂, 400.4 [M + H]⁺ |
| | 2-({(1S*)-1-[3-(1,5-dimethyl-1H- | |
| | pyrrolo[2,3-c]pyridin-3-yl)-5- | |
| | methylphenyl]ethyl}amino)benzoic | |
| | acid | |
| 27 Scheme I | 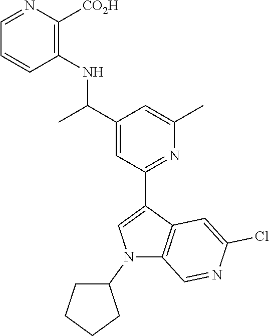 [SMILES: Cc1cc(C(C)Nc2cccnc2C(=O)O)cc(-c2cn(C3CCCC3)c3cnc(Cl)cc23)n1] | LCMS m/z (ESI) for C₂₆H₂₆ClN₅O₂, 476.3 [M + H]⁺ |
| | 3-({1-[2-(5-chloro-1-cyclopentyl-1H- | |
| | pyrrolo[2,3-c]pyridin-3-yl)-6- | |
| | methylpyridin-4- | |
| | yl]ethyl}amino)pyridine-2-carboxylic | |
| | acid | |
| 28 Scheme II | 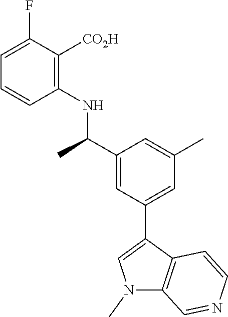 [SMILES: Cc1cc(-c2cn(C)c3cnccc23)cc([C@@H](C)Nc2cccc(F)c2C(=O)O)c1] | ¹H NMR (400 MHz, DMSO-d₆): 8.88 (d, J = 0.6 Hz, 1H), 8.16 (d, J = 5.5 Hz, 1H), 8.15-8.07 (m, 1H), 7.92 (s, 1H), 7.65 (dd, J = 0.9, 5.6 Hz, 1H), 7.43 (s, 1H), 7.33 (s, 1H), 7.18 (dt, J = 6.3, 8.3 Hz, 1H), 7.06 (s, 1H), 6.44-6.27 (m, 2H), 4.71 (br d, J = 5.8 Hz, 1H), 3.94 (s, 3H), 2.35 (s, 3H), 1.52 (d, J = 6.6 Hz, 3H) LCMS m/z (ESI) for C₂₄H₂₂FN₃O₂, 404.3 [M + H]⁺ |
| | 2-fluoro-6-({(1R)-1-[3-methyl-5-(1- | |
| | methyl-1H-pyrrolo[2,3-c]pyridin-3- | |
| | yl)phenyl]ethyl}amino)benzoic acid | |
| 29 Scheme I | 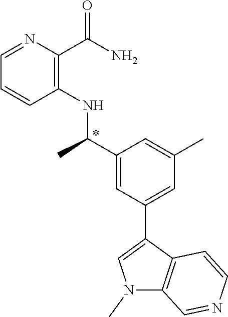 [SMILES: Cc1cc(-c2cn(C)c3cnccc23)cc([C@@H](C)Nc2cccnc2C(N)=O)c1] | Peak 2: ¹H NMR (400 MHz, DMSO-d₆): 8.88 (d, J = 0.6 Hz, 1H), 8.16 (d, J = 5.5 Hz, 1H), 8.15-8.07 (m, 1H), 7.92 (s, 1H), 7.65 (dd, J = 0.9, 5.6 Hz, 1H), 7.43 (s, 1H), 7.33 (s, 1H), 7.18 (dt, J = 6.3, 8.3 Hz, 1H), 7.06 (s, 1H), 6.44-6.27 (m, 2H), 4.71 (br d, J = 5.8 Hz, 1H), 3.94 (s, 3H), 2.35 (s, 3H), 1.52 (d, J = 6.6 Hz, 3H) LCMS m/z (ESI) for C₂₃H₂₃N₅O, 386.2 [M + H]⁺ [α]_{D}³³ = −363° (c 0.001, MeOH) |
| | 3-({(1R*)-1-[3-methyl-5-(1-methyl- | |
| | 1H-pyrrolo[2,3-d]pyridin-3- | |
| | yl)phenyl]ethyl}amino)pyridine-2- | |
| | carboxamide | |

### Example X1

The compounds provided in Table X1 are some prophetic deuterated analogs (PDA) of Example 1. The Formula (X1) is a generic formula of deuterated Example 1, wherein Y^{1a}, Y^{1b}, Y^{1c}, Y^{2a}, Y^{2b}, Y³, and Y⁴ are each independently H or D (deuterium) and wherein at least one of them is D. The deuterated analogs of Example X1 in Table X1 can be predicted based on the metabolic profile of Example 1, with MetaSite (moldiscovery.com/software/metasite/). Y^{1a}, Y^{1b}, Y^{1c}, Y^{2a}, Y^{2b}, Y³, and Y⁴ are predicted metabolized positions based on MetaSite predictions.

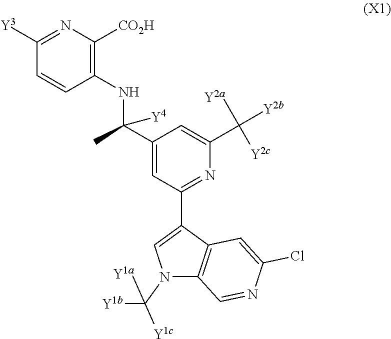

TABLE X1

| PDA # | Y^{1a}-Y^{1c} | Y^{2a}-Y^{2c} | Y³ | Y⁴ |
|---|---|---|---|---|
| X1-1 | D | H | H | H |
| X1-2 | H | D | H | H |
| X1-3 | H | H | D | H |
| X1-4 | H | H | H | D |
| X1-5 | D | D | H | H |
| X1-6 | D | H | D | H |
| X1-7 | D | H | H | D |
| X1-8 | H | D | D | H |
| X1-9 | H | D | H | D |
| X1-10 | H | H | D | D |

### Example X5

The compounds provided in Table X5 are some prophetic deuterated analogs (PDA) of Example 5. The Formula (X5) is a generic formula of deuterated Example 5, wherein Y^{1a}, Y^{1b}, Y^{1c}, Y^{2a}, Y^{2b}, Y³, and Y⁴ are each independently H or D (deuterium) and wherein at least one of them is D. The deuterated analogs of Example X5 in Table X5 can be predicted based on the metabolic profile of Example 5, with MetaSite (moldiscovery.com/software/metasite/). Y^{1a}, Y^{1b}, Y^{1c}, Y^{2a}, Y^{2b}, Y³, and Y⁴ are predicted metabolized positions based on MetaSite predictions.

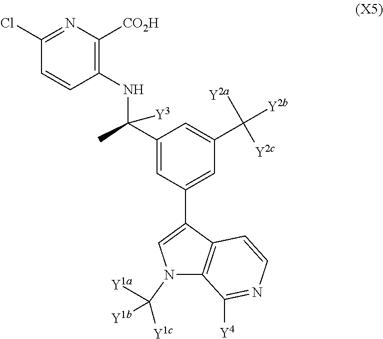

TABLE X5

| PDA # | Y^{1a}-Y^{1c} | Y^{2a}-Y^{2c} | Y³ | Y⁴ |
|---|---|---|---|---|
| X1-1 | D | H | H | H |
| X1-2 | H | D | H | H |
| X1-3 | H | H | D | H |
| X1-4 | H | H | H | D |
| X1-5 | D | D | H | H |
| X1-6 | D | H | D | H |
| X1-7 | D | H | H | D |
| X1-8 | H | D | D | H |
| X1-9 | H | D | H | D |
| X1-10 | H | H | D | D |

### Example X8

The compounds provided in Table X8 are some prophetic deuterated analogs (PDA) of Example 8. The Formula (X8) is a generic formula of deuterated Example 8, wherein Y^{1a}, Y^{1b}, Y^{1c}, Y^{2a}, Y^{2b}, Y^{3a}, Y^{3b}, Y^{3c}, and Y⁴ are each independently H or D (deuterium) and wherein at least one of them is D. The deuterated analogs of Example X8 in Table X8 can be predicted based on the metabolic profile of Example 8, with MetaSite (moldiscovery.com/software/metasite/). Y^{1a}, Y^{1b}, Y^{1c}, Y^{2a}, Y^{2b}, Y^{3a}, Y^{3b}, Y^{3c}, and Y⁴ are predicted metabolized positions based on MetaSite predictions.

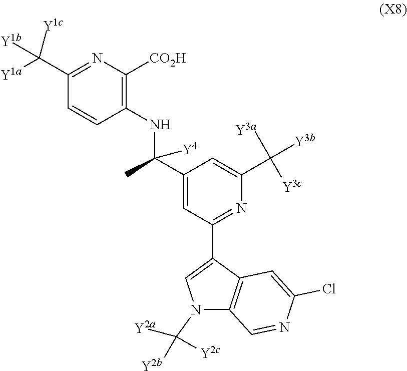

TABLE X8

| PDA # | Y^{1a}-Y^{1c} | Y^{2a}-Y^{2c} | Y^{3a}-Y^{3c} | Y⁴ |
|---|---|---|---|---|
| X1-1 | D | H | H | H |
| X1-2 | H | D | H | H |
| X1-3 | H | H | D | H |
| X1-4 | H | H | H | D |
| X1-5 | D | D | H | H |
| X1-6 | D | H | D | H |
| X1-7 | D | H | H | D |
| X1-8 | H | D | D | H |
| X1-9 | H | D | H | D |
| X1-10 | H | H | D | D |

### Example X9

The compounds provided in Table X9 are some prophetic deuterated analogs (PDA) of Example 9. The Formula (X9) is a generic formula of deuterated Example 9, wherein Y^{1a}, Y^{1b}, Y^{1c}, Y^{2a}, Y^{2b}, Y^{2c}, Y^{3a}, Y^{3b}, Y^{3c}, Y⁴, and Y⁵ are each independently H or D (deuterium) and wherein at least one of them is D. The deuterated analogs of Example X9 in Table X9 can be predicted based on the metabolic profile of Example 9, with MetaSite (moldiscovery.com/software/metasite). Y^{1a}, Y^{1b}, Y^{1c}, Y^{2a}, Y^{2b}, Y^{2c}, Y^{3a}, Y^{3b}, Y^{3c}, Y⁴, and Y⁵ are predicted metabolized positions based on MetaSite predictions.

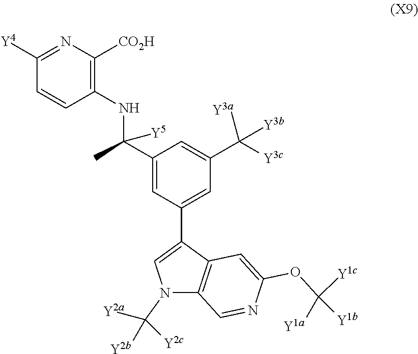

TABLE X9

| PDA # | Y^{1a}-Y^{1c} | Y^{2a}-Y^{2c} | Y^{3a}-Y^{3c} | Y⁴ | Y⁵ |
|---|---|---|---|---|---|
| X1-1 | D | H | H | H | H |
| X1-2 | H | D | H | H | H |
| X1-3 | H | H | D | H | H |
| X1-4 | H | H | H | D | H |
| X1-5 | H | H | H | H | D |
| X1-6 | D | D | H | H | H |
| X1-7 | D | H | D | H | H |
| X1-8 | D | H | H | D | H |
| X1-9 | D | H | H | H | D |
| X1-10 | H | D | D | H | H |
| X1-11 | H | D | H | D | H |
| X1-12 | H | D | H | H | D |
| X1-13 | H | H | D | D | H |
| X1-14 | H | H | D | H | D |
| X1-15 | H | H | H | D | D |

General methods/reviews of obtaining metabolite profile and identifying metabolites of a compound are described in: DAVLIE, D., et al., “Assessment of Three Human in Vitro Systems in the Generation of Major Human Excretory and Circulating Metabolites,” Chemical Research in Toxicology, 2009, 22(2):357-368; KING, R., “Chapter Three—Biotransformations in Drug Metabolism.” Drug Metabolism Handbook: Concepts and Applications in Cancer Research, edited by Ala F. Nassar et al., Wiley, 2022, 17-38; WU, Y., et al., “Metabolite Identification in Preclinical and Clinical Phase of Drug Development,” Current Drug Metabolism, 2021, 22(11):838-857; GODZIEN, J., et al., “Chapter Fifteen—Metabolite Annotation and Identification.” Comprehensive Analytical Chemistry, Elsevier, 2018, 415-445.

Numerous publicly available and commercially available software tools are available to aid in the predictions of metabolic pathways and metabolites of compounds. Examples of such tools include, BioTransformer 3.0 (BIOTRANSFORMER 3.0, “Metabolism Prediction,” www.biotransformer.ca. Retrieved Jul. 18, 2024, from URL biotransformer.ca/new) which predicts the metabolic biotransformations of small molecules using a database of known metabolic reactions; MetaSite (MOLECULAR DISCOVERY LTD., “MetaSite,” www.moldiscovery.com. Retrieved Jul. 18, 2024, from URL moldiscovery.com/software/metasite/) which predicts metabolic transformations related to cytochrome P450 and flavin-containing monooxygenase mediated reactions in phase I metabolism; and Lhasa Meteor Nexus (LHASA LIMITED, “Metabolite identification and analysis,” www.lhasalimited.org. Retrieved Jul. 18, 2024, from URL Ihasalimited.org/products/meteor-nexus.htm) offers prediction of metabolic pathways and metabolite structures using a range of machine learning models, which covers phase I and phase II biotransformations of small molecules.

Examples X1, X5, X8, and X9 (PDAs of Examples 1, 5, 8, and 9) in Tables X1, X5, X8, and X9 may afford certain therapeutic advantages resulting from greater metabolic stability, for example increased in vivo half-life, reduced dosage requirements, reduced CYP450 inhibition (competitive or time dependent), or an improvement in therapeutic index or tolerability.

### Biological Assays and Data

pAKT-1(SER 473) HTRF Assay—SKBR3 Isogenic Cell Lines

The human epithelial breast cell SKBR3 containing the PIK3CA H1047R hotspot mutation is used as the host model for this assay. PI3Ka-driven decrease in activation of the AKT-1 is monitored by phosphorylation of its serine residue at position 473 using a commercial HTRF assay (CisBio).

Briefly, SKBR3-H107R mutant cells are seeded at 10,000 cells/well in 40 μL of growth medium and rested overnight at 37° C., 5% CO₂. The following day, compounds are plated in 10 doses of 1:3 DMSO serial dilutions and promptly diluted in cell culture medium at 500 UM top dose. Media-diluted compounds are transferred to the rested cells at 10 UM final top dose, and then incubated for 2 hours at 37° C., 5% CO₂.

After compound treatment, the medium is removed from the plates and 50 μL of lysis buffer is added to each well. Lysates are transferred to low volume 384-well plates containing fluorescent labeled antibodies and incubated overnight at room temperature protected from light according to manufacturer's protocol.

Plates are read in HTRF compatible reader and IC₅₀ values for each compound are calculated using ActivityBase software.

### Phosphoinositide 3-Kinase Alpha (PI3Kα) H1047R Protein Production

The nucleotide sequence encoding human recombinant full-length (FL) H1047R mutant p110α catalytic subunit (amino acids 1-1068) of PIK3α, containing N-terminal FLAG-TEV-Avi tag, and FL p85α regulatory subunit were codon optimized, custom synthesized at GenScript Biotech Corp. (Piscataway, NJ) and cloned into pFASTBAC-Dual vector. Mutant p110α were cloned after the polyhedrin promoter, and p85α was cloned into the same vector after p10 promoter. This construct was used to make baculovirus (Thermo Fisher Scientific, Madison, WI). Protein complex of mutant p110α was co-expressed with p85α at Pfizer, Inc., La Jolla facility. Protein was expressed by infecting Sf21 cells with multiplicity of infection=1 of mutant PIK3a baculovirus. Cells were grown at 27° C. for 72 hours in ESF-921 medium (Expression Systems, Davis, CA) and lysed in the lysis buffer (LB), containing 50 mM Tris (pH 8.0), 200 mM NaCl and 0.25 mM tris(2-carboxyethyl) phosphine. The lysate was spun at 20,000×g for 1 hour. Lysate was pre-bound to Pierce™ Anti-DYKDDDDK Affinity Resin (Thermo Fisher Scientific, Inc.) in batch mode, rotating for 3 hours at 4° C. Resin was placed in gravity column and washed with 20×CV of LB. Protein was eluted with 3×CV of LB, containing 0.2 mg/ml commercial FLAG peptide. The protein was concentrated and purified over Superdex 200 column (Cytiva, Marlborough, MA). Protein complex peak fractions were pooled, and flash frozen in aliquots in liquid nitrogen.

### PI3Kα H1047R Biochemical Assay

Human recombinant FL H1047R mutant of PIK3α (p110α H1047R/p85α) enzyme, containing N-terminal FLAG-TEV-Avi tag on the p110α catalytic subunit, was produced as described above and assayed at Pfizer, Inc, La Jolla facility, using ADP-Glo assay kit (Promega Corp., Madison, WI). Test compounds were plated in 11 doses of 1:3 serial dilutions (100 nL volume in 100% DMSO per well) on a 384-well plate. Enzyme assay buffer (5 μL), containing 100 mM HEPES (pH 7.5), 100 mM NaCl, 20 mM MgCl₂, 10 mM dithiothreitol, 0.03% CHAPS detergent, and 6 nM PIK3a H1047R enzyme was added to each well. The plate was briefly spun at 800 rpm and preincubated for 60 minutes on a shaker plate. Substrate mix (5 μL), containing 100 μM Promega Ultra Pure ATP and 100 μM 08:0 PI(4,5)P2 (1,2-dioctanoyl-sn-glycero-3-phospho-(1′-myo-inositol-4′,5′-bisphosphate) (ammonium salt), Avanti Polar Lipids, Inc., Alabaster, AL) was added to each well to start the reaction, the plate was briefly spun at 800 rpm and incubated for 40 minutes at controlled room temperature. The final reactions contained 3 nM enzyme, 50 μM 08:0 PI(4,5)P2, 50 μM ATP, 50 mM NaCl, 10 mM MgCl₂, 5 mM dithiothreitol, 0.015% CHAPS detergent in 50 mM HEPES (pH 7.5). The reaction was stopped with ADP-Glo reagent, briefly spun at 800 rpm and incubated for 40 minutes. ADP-Glo Kinase Detection Reagent was added, and the plate was briefly spun at 800 rpm and incubated for 30 minutes. The luminescence was measured in a PHERAstar FSX plate reader (BMG Labtech, Inc., Cary, NC). The extents of reactions were estimated to be <20%. The 50% inhibitory potencies (IC₅₀) of test compounds were calculated by fitting the luminescence based fractional initial velocities to the 4-parameter IC₅₀ equation using ABase software (IDBS, London, United Kingdom).

Biological assay data is provided in Table 2 below. The reported IC₅₀ may be from a single assay replicate or the mean of replicates.

TABLE 2

| | Biological Assay 1 | Biological Assay 2 |
|---|---|---|
| Example | H1047R biochemical | H1047R pAKT |
| Number | IC₅₀ (nM) | IC₅₀ (nM) |
| 1 | 77 | 245 |
| 2 | 110 | 852 |
| 3 | 25 | 265 |
| 4 | 34 | 314 |
| 5 | 13 | 53 |
| 6 | 169 | 496 |
| 7 | 449 | >30,000 |
| 8 | 72 | 434 |
| 9 | 23 | 256 |
| 10 | 119 | 14,638 |
| 11 | 25 | 117 |
| 12 | 1,118 | 1,516 |
| 13 | 671 | 1,145 |
| 14 | 229 | |
| 15 | 323 | 777 |
| 16 | 146 | 177 |
| 17 | 171 | ≥27,969 |
| 18 | 187 | 117 |
| 19 | 75 | 969 |
| 20 | 64 | 846 |
| 21 | 213 | 215 |
| 22 | 778 | 1,389 |
| 23 | 13 | 3,226 |
| 24 | 28 | 767 |
| 25 | 152 | 262 |
| 26 | 39 | 345 |
| 27 | 61 | 143 |
| 28 | 22 | 522 |
| 29 | 147 | 311 |

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

We claim:

1. A compound of Formula (I): 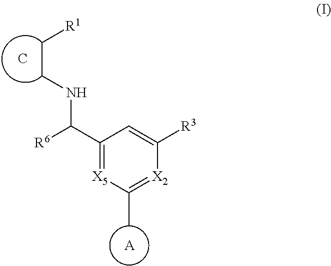
or a pharmaceutically acceptable salt thereof, wherein:
Ring A is a nine or ten membered bicyclic heteroaryl containing one, two or three heteroatoms selected from the group consisting of N, O and S, wherein the heteroaryl is optionally substituted with one or two groups independently selected from the group consisting of halogen, C₁-C₆ alkyl, C₁-C₆ alkoxy, C₃-C₆ cycloalkyl, and NR^{a}R^{b};
Ring C is phenyl or a 5 or 6 membered heteroaryl group containing one or two heteroatoms independently selected from N and S, wherein Ring C is optionally substituted by R²;
X₂ and X₅ are independently selected from the group consisting of N and CH;
R¹ is selected from the group consisting of —C(═O) OH and a carboxylic acid isostere;
R² is selected from the group consisting of hydrogen, halogen, C₁-C₃ alkyl, and C₁-C₃ fluoroalkyl;
R³ is selected from the group consisting of halogen, C₁-C₃ alkyl, and C₁-C₃ haloalkyl;
R⁶ is hydrogen or methyl; and
R^{a} and R^{b} are independently selected from hydrogen and C₁-C₃ alkyl.

2. The compound of claim 1, or a pharmaceutically acceptable salt thereof, wherein X₂ is N and X₅ is CH.

3. The compound of claim 1, or a pharmaceutically acceptable salt thereof, wherein R³ is methyl.

4. The compound of claim 1, or a pharmaceutically acceptable salt thereof, wherein R⁶ is methyl.

5. The compound of claim 1, or a pharmaceutically acceptable salt thereof, having Formula (Ia):

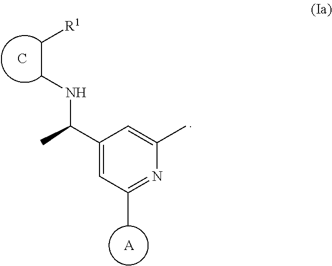

6. The compound of claim 6, or a pharmaceutically acceptable salt thereof, wherein Ring A is selected from the group consisting of: 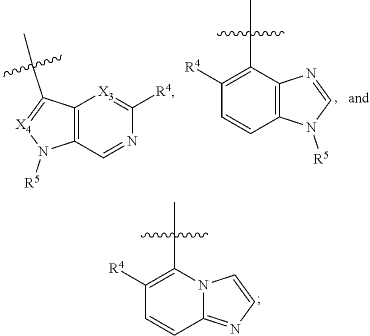
wherein X₃ and X₄ are independently selected from N and CH, wherein only one of X₃ and X₄ may be N;
R⁴ is selected from the group consisting of selected from the group consisting of hydrogen, halogen, C₁-C₆ alkyl, C₁-C₆ alkoxy, NH₂ and NH(CH₃); and
R⁵ is selected from the group consisting of hydrogen, C₁-C₆ alkyl, and C₃-C₆ cycloalkyl.

7. The compound of claim 1, or a pharmaceutically acceptable salt thereof, wherein Ring A is:

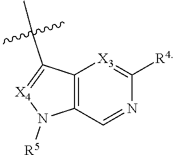

8. The compound of claim 7, or a pharmaceutically acceptable salt thereof, wherein R⁴ is selected from the group consisting of hydrogen, chloro, methyl, and methoxy.

9. The compound of claim 7, or a pharmaceutically acceptable salt thereof, wherein R⁵ is selected from the group consisting of hydrogen, methyl, ethyl, isopropyl, and cyclopentyl.

10. The compound of claim 1, or a pharmaceutically acceptable salt thereof, wherein Ring C is selected from the group consisting of:

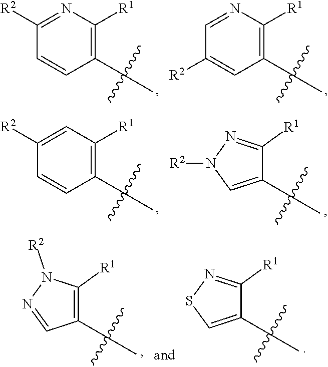

11. The compound of claim 10, or a pharmaceutically acceptable salt thereof, wherein R² is selected from the group consisting of hydrogen, Cl, F, methyl, and difluoromethyl.

12. The compound of claim 10, or a pharmaceutically acceptable salt thereof, wherein R¹ is —C(═O)OH.

13. The compound of claim 1, selected from the group consisting of:
3-({(1R*)-1-[2-(5-chloro-1-methyl-1H-pyrrolo[2,3-c]pyridin-3-yl)-6-methylpyridin-4-yl]ethyl}amino)pyridine-2-carboxylic acid;
2-({(1R*)-1-[2-methyl-6-(1-methyl-1H-pyrrolo[2,3-c]pyridin-3-yl) pyridin-4-yl]ethyl}amino)benzoic acid;
2-({(1R*)-1-[3-methyl-5-(1-methyl-1H-pyrrolo[2,3-c]pyridin-3-yl)phenyl]ethyl}amino)benzoic acid;
3-({(1R*)-1-[3-methyl-5-(1-methyl-1H-pyrrolo[2,3-c]pyridin-3-yl)phenyl]ethyl}amino)pyridine-2-carboxylic acid;
6-chloro-3-({(1R*)-1-[3-methyl-5-(1-methyl-1H-pyrrolo[2,3-c]pyridin-3-yl)phenyl]ethyl}amino)pyridine-2-carboxylic acid; and
3-({(1R*)-1-[2-methyl-6-(1-methyl-1H-pyrrolo[2,3-c]pyridin-3-yl) pyridin-4-yl]ethyl}amino)pyridine-2-carboxylic acid;
4-({1-[2-(5-chloro-1-methyl-1H-pyrrolo[2,3-c]pyridin-3-yl)-6-methylpyridin-4-yl]ethyl}amino)-1-methyl-1H-pyrazole-3-carboxylic acid;
3-({(1R)-1-[2-(5-chloro-1-methyl-1H-pyrrolo[2,3-c]pyridin-3-yl)-6-methylpyridin-4-yl]ethyl}amino)-6-methylpyridine-2-carboxylic acid;
2-({(1R*)-1-[3-(5-methoxy-1-methyl-1H-pyrrolo[2,3-c]pyridin-3-yl)-5-methylphenyl]ethyl}amino)benzoic acid;
N-(methanesulfonyl)-2-({(1R)-1-[3-methyl-5-(1-methyl-1H-pyrrolo[2,3-c]pyridin-3-yl)phenyl]ethyl}amino)benzamide;
2-({(1R)-1-[2-(5-chloro-1-methyl-1H-pyrrolo[2,3-c]pyridin-3-yl)-6-methylpyridin-4-yl]ethyl}amino)-5-fluorobenzoic acid;
3-({[2-(5-chloro-1-methyl-1H-pyrrolo[2,3-c]pyridin-3-yl)-6-methylpyridin-4-yl]methyl}amino)pyridine-2-carboxylic acid;
3-({(1R)-1-[2-(5-chloro-1-methyl-1H-pyrrolo[2,3-c]pyridin-3-yl)-6-methylpyridin-4-yl]ethyl}amino)pyridine-2-carboxamide;
4-({1-[2-(5-chloro-1-methyl-1H-pyrrolo[2,3-c]pyridin-3-yl)-6-methylpyridin-4-yl]ethyl}amino)-1-methyl-1H-pyrazole-5-carboxylic acid;
3-({1-[2-(5-chloro-1-ethyl-1H-pyrrolo[2,3-c]pyridin-3-yl)-6-methylpyridin-4-yl]ethyl}amino)pyridine-2-carboxylic acid;
3-({1-[6-(5-chloro-1-methyl-1H-pyrrolo[2,3-c]pyridin-3-yl)-4-methylpyridin-2-yl]ethyl}amino)pyridine-2-carboxylic acid;
4-({(1R*)-1-[2-(5-chloro-1-methyl-1H-pyrrolo[2,3-c]pyridin-3-yl)-6-methylpyridin-4-yl]ethyl}amino)-1-(difluoromethyl)-1H-pyrazole-3-carboxylic acid;
3-({1-[2-(5-chloro-1-methyl-1H-pyrrolo[2,3-c]pyridin-3-yl)-6-methylpyrimidin-4-yl]ethyl}amino)pyridine-2-carboxylic acid;
3-({(1R)-1-[3-methyl-5-(1-methyl-1H-pyrazolo[3,4-c]pyridin-3-yl)phenyl]ethyl}amino)pyridine-2-carboxamide acid;
3-({(1R)-1-[2-(5-chloro-1-methyl-1H-pyrrolo[2,3-c]pyridin-3-yl)-6-methylpyridin-4-yl]ethyl}amino)-5-fluoropyridine-2-carboxylic acid;
3-({1-[2-(1H-1,3-benzimidazol-4-yl)-6-methylpyridin-4-yl]ethyl}amino)pyridine-2-carboxylic acid;
3-({(1R)-1-[2-(imidazo[1,2-a]pyridin-5-yl)-6-methylpyridin-4-yl]ethyl}amino)pyridine-2-carboxylic acid;
4-({(1S)-1-[2-(5-chloro-1-methyl-1H-pyrrolo[2,3-c]pyridin-3-yl)-6-methylpyridin-4-yl]ethyl}amino)-1,2-thiazole-3-carboxylic acid;
2-({(1S*)-1-[3-methyl-5-(5-methyl-5H-pyrrolo[3,2-d]pyrimidin-7-yl)phenyl]ethyl}amino)benzoic acid;
3-[(1-{2-[5-chloro-1-(propan-2-yl)-1H-pyrrolo[2,3-c]pyridin-3-yl]-6-methylpyridin-4-yl}ethyl)amino]pyridine-2-carboxylic acid;
2-({(1S*)-1-[3-(1,5-dimethyl-1H-pyrrolo[2,3-c]pyridin-3-yl)-5-methylphenyl]ethyl}amino)benzoic acid;
3-({1-[2-(5-chloro-1-cyclopentyl-1H-pyrrolo[2,3-c]pyridin-3-yl)-6-methylpyridin-4-yl]ethyl}amino)pyridine-2-carboxylic acid;
2-fluoro-6-({(1R)-1-[3-methyl-5-(1-methyl-1H-pyrrolo[2,3-c]pyridin-3-yl)phenyl]ethyl}amino)benzoic acid; and
3-({(1R*)-1-[3-methyl-5-(1-methyl-1H-pyrrolo[2,3-c]pyridin-3-yl)phenyl]ethyl}amino)pyridine-2-carboxamide;
or a pharmaceutically acceptable salt thereof.

14. A pharmaceutical composition comprising the compound of claim 1, or a pharmaceutically acceptable salt thereof, and at least one pharmaceutically acceptable excipient.

15. A pharmaceutical composition comprising the compound of claim 13, or a pharmaceutically acceptable salt thereof, and at least one pharmaceutically acceptable excipient.

16. A method for treating cancer, comprising administering to a subject in need thereof a therapeutically effective amount of the compound of claim 1, or a pharmaceutically acceptable salt thereof.

17. A method for treating cancer, comprising administering to a subject in need thereof a therapeutically effective amount of the compound of claim 13, or a pharmaceutically acceptable salt thereof.

18. A method for treating cancer, comprising administering to a subject in need thereof a therapeutically effective amount of the compound of claim 1, or a pharmaceutically acceptable salt thereof, and further comprising administering an amount of an additional anti-cancer therapeutic agent.

19. A pharmaceutical composition comprising a compound of claim 1 or a pharmaceutically acceptable salt thereof, and an anti-cancer agent or a pharmaceutically acceptable salt thereof, and an excipient.

20. A pharmaceutical composition comprising a compound of claim 13 or a pharmaceutically acceptable salt thereof, and an anti-cancer agent or a pharmaceutically acceptable salt thereof, and an excipient.
